(19) United States
(12) Patent Application Publication
(10) Pub. No.: US 2025/0145968 A1
(43) Pub. Date: May 8, 2025

(54) GENETIC ENGINEERING OF BACTERIOPHAGES USING CRISPR-CAS13A

(71) Applicant: The Regents of the University of California, Oakland, CA (US)

(72) Inventors: Joseph Bondy-Denomy, Oakland, CA (US); Jingwen Guan, Oakland, CA (US); Senen Mendoza, Oakland, CA (US)

(73) Assignee: The Regents of the University of California, Oakland, CA (US)

(21) Appl. No.: 18/837,544

(22) PCT Filed: Feb. 8, 2023
(86) PCT No.: PCT/US2023/062188

§ 371 Date: Aug. 9, 2024

Related U.S. Application Data
(60) Provisional application No. 63/308,745, filed on Feb. 10, 2022.

(51) Int. Cl. C12N 7/00; C12N 9/22; C12N 15/11; C12N 15/63

(52) U.S. Cl. CPC C12N 7/00; C12N 9/22; C12N 15/11; C12N 15/63; C12N 2310/20; C12N 2795/00021; C12N 2795/00022; C12N 2795/00052

(57) ABSTRACT

The present disclosure provides methods and compositions for modifying the genomes of bacteriophages, in particular by integrating a coding sequence for an anti-CRISPR protein into the bacteriophage genome together with a desired modification such as a deletion, insertion, or nucleotide substitution, and selecting for bacteriophages with the integrated anti-CRISPR coding sequence by introducing the phage into a counter selection strain comprising an RNA-targeting CRISPR-Cas system.

### CROSS-REFERENCE TO RELATED APPLICATIONS

This application is a 371 U.S. National Phase Application of PCT/US2023/062188, filed Feb. 8, 2023, which claims benefit of priority to U.S. provisional application 63/308,745, filed Feb. 10, 2022, both of which are is herein incorporated by reference in their entirety for all purposes.

### SEQUENCE LISTING

A Sequence Listing conforming to the rules of WIPO Standard ST.26 is hereby incorporated by reference. Said Sequence Listing has been filed as an electronic document via Patent Center encoded as XML in UTF-8 text. The electronic document, created on Jun. 5, 2023, is entitled “081906-1361930-246110PC_ST26.xml”, and is 151,049 bytes in size.

### BACKGROUND OF THE INVENTION

Bacteriophages are viruses that infect bacteria and can cause their lysis after replication. In recent decades, the rapid emergence of multi-antibiotic resistant bacterial pathogens and simultaneous decline in the discovery of new antibiotics has rekindled interest in the use of phages as alternative antimicrobial therapeutics (phage therapy) [1, 2]. Phages offer many advantages over antibiotics, including high specificity and efficient self-propagation in the presence of their bacterial host [3-5]. However, host range limitations and the rapid emergence of phage resistance in clinical strains become barriers for further exploiting phage therapy [1, 3, 6]. Phage genome engineering may help overcome these hurdles [7, 8]. Robust phage engineering tools will aid fundamental discoveries, broaden host range, enhance evasion of host antiviral defense systems, and reduce phage toxicity and immunogenicity [9-12]. Phage engineering techniques often utilize homologous recombination (HR) with a template plasmid [13, 14], coupled with a selective pressure such as CRISPR-Cas targeting. CRISPR-Cas systems (clustered regularly interspaced short palindromic repeats and CRISPR-associated proteins), are adaptive anti-phage immune systems in prokaryotes [15, 16]. CRISPR-Cas programmable targeting enables effective selection for phage recombinants by removing wild-type phages from the population.

To date, all CRISPR-based screening tools applied for phage engineering recognize and target phage genomic DNA. However, as a consequence of the everlasting evolutionary battle between bacteria and phages in nature, phages have amassed various strategies to circumvent DNA-targeting immunity, including anti-CRISPR proteins, DNA base modifications, and genome segregation [17, 18]. For example, P. aeruginosa phage ΦKZ is resistant to a broad spectrum of DNA-targeting immune systems via assembly of a proteinaceous “nucleus-like” (phage nucleus) structure to shield phage DNA during infection [19, 20]. No genetic tools are available for this phage family, which possesses numerous exciting biological features that have only been studied to date with the plasmid-based over-expression system [21]. Despite the failure of DNA-targeting CRISPR-Cas systems, the mRNA-targeting CRISPR-Cas13a system (type VI-A) [22] effectively inhibits ΦKZ replication by degrading phage mRNA that is exported out of the phage nucleus to the cytoplasm [19].

### BRIEF SUMMARY OF THE INVENTION

In some embodiments, a method of introducing a genetic modification at a locus within the genome of a bacteriophage is provided. In some embodiments, the method comprises:
i) providing a first population of bacterial cells comprising a polynucleotide and the bacteriophage, wherein the polynucleotide comprises a coding sequence for an anti-CRISPR protein that inhibits an RNA-targeting CRISPR-Cas system in the bacterial cells, flanked by two homology arms comprising nucleotide sequence homology to the locus;
ii) culturing the bacterial cells under conditions conducive to homologous recombination between the polynucleotide and the bacteriophage genome, and obtaining a lysate from the cultured cells.

In some embodiments, the method further comprises:
iii) infecting a second population of bacterial cells comprising the RNA-targeting CRISPR-Cas system with the lysate; and
iv) isolating bacteriophage from the contacted population of CRISPR bacterial cells.

In some embodiments, the bacteriophage is a lytic bacteriophage and the isolating comprises isolating lytic plaques from the contacted population of CRISPR bacterial cells.

In some embodiments, the method further comprises verifying the integration of the coding sequence for the CRISPR inhibitor at the locus within the bacteriophage genome.

In some embodiments, the RNA-targeting CRISPR-Cas system comprises Cas13a.

In some embodiments, one or both of the homology arms are at least about 50, 100, 150, 200, 250, 300, 350, 400, 450, 500, 550, 600, or more nucleotides in length.

In some embodiments, the bacteriophage is selected from the group consisting of ΦKZ, OMKO1, and PaMx41.

In some embodiments, the integration of the CRISPR inhibitor into the bacteriophage genome at the locus introduces a genetic modification at the locus. In some embodiments, the genetic modification is a deletion. In some embodiments, the genetic modification is an insertion. In some embodiments, the genetic modification is a nucleotide substitution.

In some embodiments, the RNA-targeting CRISPR-Cas system comprises a crRNA comprising one or more mismatches between two direct repeat sequences and/or a truncation of 1, 2, 3, 4, 5, 6, 7, 8, 9, 10 or more nucleotides within a direct repeat at the 5′ and/or 3′ end of the crRNA.

In some embodiments, the RNA-targeting CRISPR-Cas system comprises a crRNA containing one direct repeat sequence.

In some embodiments, the anti-CRISPR protein is AcrVIA1.

In some embodiments, the crRNA targets orf120 or orf146 (e.g., from phiKZ phage), or an ortholog thereof. In some embodiments, the locus is from phiKZ phage and is selected from the group consisting of orf39, orf54, orf120, orf146, orf93, orf241, orf242, orf89, orf90, orf91, orf92, orf93, and orthologs thereof.

In some embodiments, the first population of bacterial cells comprises the bacteriophage prior to comprising the polynucleotide. In some embodiments, the first population of bacterial cells comprises the bacteriophage subsequent to comprising the polynucleotide.

In some embodiments, the polynucleotide and flanking homology arms are present in an editing plasmid, and wherein the editing plasmid is introduced into the first population of bacterial cells by electroporation.

In some embodiments, the second population of bacterial cells comprises the RNA-targeting CRISPR-Cas system prior to being contacted with the lysate. In some embodiments, the second population of bacterial cells comprises the RNA-targeting CRISPR-Cas system subsequent to being contacted with the lysate.

Also provided is a modified bacteriophage produced using the method as described above or elsewhere herein. Also provided is a bacterial cell comprising the modified bacteriophage as described above.

Also provided is a modified bacteriophage comprising a coding sequence for an anti-CRISPR protein integrated into the bacteriophage genome at a locus that is not essential for phage replication, wherein the anti-CRISPR protein can inhibit an RNA-targeting CRISPR-Cas system, and wherein the locus also comprises a deletion, insertion, or mutation relative to an otherwise equivalent, non-modified bacteriophage. In some embodiments, the anti-CRISPR protein is AcrVIA1. In some embodiments, the bacteriophage is selected from the group consisting of ΦKZ, OMKO1, and PaMx41. In some embodiments, the locus is selected from the group consisting of orf39, orf54, orf120, orf146, orf93, orf241, orf242, orf89, orf90, orf91, orf92, orf93, and orthologs thereof.

Also provided is a bacterial cell comprising the modified bacteriophage as described above.

Also provided is a bacterial cell comprising a polynucleotide and a bacteriophage, wherein the polynucleotide comprises a coding sequence for an anti-CRISPR protein that inhibits an RNA-targeting CRISPR-Cas system, flanked by two homology arms comprising substantial nucleotide sequence identity to a locus within the genome of the bacteriophage. In some embodiments, the anti-CRISPR protein is AcrVIA1. In some embodiments, the RNA-targeting CRISPR-Cas system comprises Cas13a.

### BRIEF DESCRIPTION OF THE DRAWINGS

FIG. 1A-C. Optimization of CRISPR-Cas13a crRNA expression vector for efficient phage interference. (A) Schematic of three versions of CRISPR-Cas13a crRNA cassette (B) Efficiency of plaquing of three versions of crRNA cassette targeting two unrelated phage strains: JBD30 and ΦKZ. Wild-type phages were spotted in ten-fold serial dilutions (left to right) on a lawn of P. aeruginosa PAO1 expressing LseCas13a from the chromosome and harboring indicated crRNA expression vectors. The cassettes carried the same spacer sequences targeting transcripts of orf38 of JBD30 and orf54 of ΦKZ, respectively. (C) Cas13a interference efficiency of distinct crRNAs against ΦKZ using Version 3 crRNA expression vector. The most efficient crRNA that targets the orf120 transcript was selected for genetic engineering of ΦKZ. All plaque assays were replicated three times yielding similar results. Table 2 shows bacterial strains and phages used in this work.

FIG. 2A-D. Screening for ΦKZ recombinants by CRISPR-Cas13a counter selection. (A) Schematic of WT ΦKZ and recombinant genomes at the editing site. The acrVIA1 gene, shown as a red rectangle, was inserted downstream of orf120, with up- and downstream of homology arms (H) indicated by blue rectangles. Green stripes represent synonymous mutations that were introduced to the homology region to prevent crRNA targeting for recombinant phages. F and R indicate forward and reverse primers, respectively, being used to confirm the insertion of acrVIA1. (B) Recombinant phages were screened by PCR using primer F1 and R. (C) Recombinant phage plaques underwent 3 rounds of purification and were further confirmed by PCR using primer F2 and R. (D) Plaque assays showing robust anti-CRISPR activities acquired by recombinant ΦKZ against distinct crRNAs, owing to the successful expression and execution of the incorporated AcrVIA1. Table 3 shows crRNA sequences used in this work. Table 4 shows examples of forward (F) and reverse (R) primer sequences flanking the described genetic loci used in this work.

FIG. 3. Absence of PhuZ causes mispositioning of the phage nucleus. Left: a representative cell infected by WT ΦKZ. Right: a representative cell infected by ΔphuZ mutant. Phage DNA is stained by DAPI and shown as blue signals. The phage nucleus is mispositioned near the cell polar region upon infection by ΔphuZ, in contrast to the WT infection where the phage nucleus is centered. Scale bar denotes 2 μm.

FIG. 4A-C. Endogenous fluorescent labeling of an inner body protein of ΦKZ. (A) Visualization of individual phage particles under the fluorescence microscope. Orf39 encoding one of the major components of ΦKZ inner body is genetically fused to mNeonGreen. Each mutant phage particle is visible as a green focus (left), due to the packaging of gp93-mNeonGreen in the capsid. ΦKZ genomic DNA is labeled by DAPI (right). MNeonGreen and DAPI signals colocalize very well and individual virions are easily distinguishable. (B) Overlay images (phase-contrast and fluorescent channels) from a time-lapse movie depicting a representative PAO1 cell being infected by a gp93-mNeonGreen mutant phage. Gp93-mNeonGreen and phage DNA are shown as green and blue signals, respectively. Red arrows point to newly assembled phage capsids, both originated on the cell inner membrane (20 min) and anchored on the phage nucleus surface for subsequent phage DNA packaging (25, 45, and 75 min). (C) Overlay images from a time-lapse movie showing a PAO1 cell being infected by a WT ΦKZ loaded with gp93-mNeonGreen fusions. Packaged gp93-mNeonGreen appears as a green focus and remains bound to phage DNA throughout the infection cycle. Scale bar denotes 2 μm.

FIGS. 5A and B. Genetic engineering of the therapeutic jumbo phage OMKO1. (A) Schematic of genomes of two engineered OMKO1 variants where the indicated gene fragments were integrated into the WT genome. OMKO1::Acr: acrVIA1 was inserted upstream of the shell gene. OMKO1::Acr-BC: acrVIA1 was inserted together with a barcode sequence (BC) downstream of the major capsid gene. (B) Plaque assays of WT OMKO1 and mutants on lawns expressing cas13 and crRNAs targeting the OMKO1 homologues of the ΦKZ orf120 (crRNA no. 1) or orf146 (crRNA no. 2) transcripts.

FIG. 6A-C. Genetic engineering of phage PaMx41. (A) Plaque assays showing PaMx41 resistance to a broad variety of DNA-targeting CRISPR-Cas systems. (B) PaMx41 exhibits significant sensitivity to CRISPR-Cas13a. Grna #5 highlighted in the red frame has been used for PaMx41 genome engineering. (C) PaMx41 Δorf24 mutant strain is resistant to diverse crRNAs of CRISPR-Cas13a, due to the expression of AcrVIA1 from the phage genome.

FIG. 7. Plaque efficiency assays of distinct crRNAs of CRISPR-Cas13a targeting transcripts of diverse ΦKZ genes. Different crRNAs show varied degrees of Cas13a interference efficiency against ΦKZ. NT, non-targeting.

FIG. 8. Sequence alignment of wild type ΦKZ and three escaper mutants at the engineered genomic site. Escaper mutants were isolated and verified by PCR and sequencing. The WT orf120 sequence is highlighted in blue and the downstream region is highlighted in grey. The stop codon (TGA) of orf120 is highlighted in green and Escaper #3 reconstitutes it to TAG. The sequence in the red frame matches the spacer sequence of the crRNA that was used to target and eliminate WT phages. Deletions were indicated by dashed lines and their corresponding numbers of absent base pairs.

FIG. 9. Determination of host range of ΦKZ ΔphuZ and Δorf93 mutants by plaque assay on P. aeruginosa clinical strains.

FIG. 10. Failure of genetic editing the shell gene (orf54) in ΦKZ. (A) Schematic of genomes of WT ΦKZ and three mutated orf54 variants, “Δorf54”, “FP-orf54”, and “orf54-FP”, at the editing site. Orf54, acrVIA1, and fluorescent protein (FP) are shown as blue, red, and green rectangles, respectively. F and R indicate forward and reverse primers, respectively, for PCR confirmation of orf54 engineering. (B) PCR confirmation of the indicated orf54 mutants using their corresponding pair of primers. All three mutants generated multiple bands, including a band in the same size as the single band produced by WT. (C) Genome alignment of WT phage with the isolated orf54 “pseudo knock-out” mutant (“Δorf54”). A gene cluster of ˜7 kbp (orf206-orf216) was missing in the mutant, likely as a result of phage packaging capacity. The majority of the editing plasmid used to generate recombinants was at the editing site, leaving the orf54 intact.

### DETAILED DESCRIPTION OF THE INVENTION

### 1. General

Practicing this invention utilizes routine techniques in the field of molecular biology. Basic texts disclosing the general methods of use in this invention include Sambrook and Russell, Molecular Cloning, A Laboratory Manual (3^{rd} ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 2. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter. In some embodiments, the promoter is a prokaryotic promoter, e.g., a promoter used to drive crRNA, anti-anti-CRISPR, or RNA-targeting CRISPR-Cas13 gene expression in prokaryotic cells. Typical prokaryotic promoters include elements such as short sequences at the −10 and −35 positions upstream from the transcription start site, such as a Pribnow box at the −10 position typically consisting of the six nucleotides TATAAT, and a sequence at the −35 position, e.g., the six nucleotides TTGACA.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of a crRNA and/or a nucleic acid sequence encoding a protein (e.g., an RNA-targeting Cas protein such as Cas13a or an anti-CRISPR protein). In some embodiments, the term refers to the production of a transcriptional and/or translational product encoded by a gene (e.g., a Cas13a gene) or a portion thereof. The level of expression of a DNA molecule in a cell may be assessed on the basis of either the amount of corresponding Mrna that is present within the cell or the amount of protein encoded by that DNA produced by the cell.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of an RNA-targeting CRISPR-Cas13 protein can have an increased stability, assembly, or activity as described herein.

The following eight groups each contain amino acids that are conservative substitutions for one another:
1) Alanine (A), Glycine (G);
2) Aspartic acid (D), Glutamic acid I;
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cystine (C), Methionine (M)
(see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93%, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. For example, polypeptides comprising an amino acid sequence substantially identical to SEQ ID NO:1 and optionally having anti-CRISPR activity and nucleic acids comprising a nucleotide sequence substantially identical to SEQ ID NO:2 is provided. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types, I, II, III, IV, V, and VI as well as many sub-types, with Class 1 including types I and III CRISPR systems, and Class 2 including types II, IV, V and VI; Class 1 subtypes include subtypes I-A to I-F, for example. See, e.g., Fonfara et al., Nature 5 32, 7600 (2016); Zetsche et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are affected by multiple Cas proteins, with Cas13 providing the endonuclease activity, whereas in class 2 systems they are all carried out by a single Cas, Cas9.

An “RNA-targeting CRISPR-Cas system” or “RNA-guided CRISPR-Cas system” refers to a Cas protein (an “RNA-targeting” or “RNA-guided” Cas protein or RNA ribonuclease) and crRNA that act together to target an RNA molecule comprising the target sequence specified in the spacer region of the crRNA. In some embodiments, the Cas protein within the RNA-targeting CRISPR-Cas system is a Cas13 ribonuclease, e.g., Cas13a, Cas13b, Cas 13c, Cas 13d, Cas13x, or Cas13y. Any of the numerous Cas13 proteins, of any family and from any source, can be used in the present methods. In particular embodiments, Cas13a is used, e.g., Cas13a from Leptotrichia. See, e.g., Mendoza et al. (2020), the entire disclosure of which is herein incorporated by reference.

The crRNAs, or CRISPR RNAs, used herein can be any crRNA that can function with an endogenous or exogenous RNA-targeting CRISPR-Cas system to direct the degradation of RNA comprising the crRNA target sequence. The crRNAs can be bound by the RNA-targeting Cas component of such a system, e.g., Cas13a. As used herein, an “RNA-targeting CRISPR-Cas system crRNA” refers to a crRNA that, when incubated together with one or more RNA-targeting Cas proteins such as Cas13a, can direct the protein to a target RNA sequence as defined by (e.g., being complementary or homologous to) the spacer sequence of the crRNA. An “RNA-targeting CRISPR-Cas13 crRNA” can also be any naturally occurring, or “wild-type,” crRNA that is present in a CRISPR array in any species with an RNA-targeting CRISPR-Cas system, or to a crRNA made using a repeat sequence from any CRISPR array from any species with an RNA-targeting CRISpR-Cas system. crRNAs comprise a spacer sequence of, e.g., <15 nucleotides, 15-20, 20-25, 25-30, or >30 nucleotides in length, or 20, 21, 22, 23, 24, 25, 26, 27, 28, 29, or 30 nucleotides in length, with homology to a targeted RNA transcript sequence at a position adjacent to a PAM sequence (e.g., 5′-TTC-3′), as well as one or more repeat sequences of, e.g., 10, 11, 12, 13, 14, 15, 16, 17, 18, 19, 20, 21, 22, 23, 24, 25, 26, 27, 28, 29, 30, 31, 32, 33, 34 or more nucleotides in length, or e.g., 20-40, 30-40, 25-35 nucleotides in length, comprising a stem-loop structure.

crRNAs can also be modified, e.g., in the stem region, the loop region, or outside of the stem-loop region, as described elsewhere herein, e.g., in a sequence that includes exchanged complementary base pairs within the stem region and modified nucleotides within the loop. For example, in crRNAs that comprise two repeats, the repeat sequences can differ from one another at one or more nucleotides, e.g., 1, 2, 3, 4, 5, 6, 7, 8, 9, 10, 11, 12, 13, 14, 15, or more nucleotides, as described in more detail elsewhere herein. crRNAs can also comprise a single repeat sequence together with the spacer sequence and can also comprise truncations within one or both repeat sequences, e.g., a truncation of 1, 2, 3, 4, 5, 6, 7, 8, 9, 10, or greater nucleotides from the 5′ or 3′ end of the crRNA, e.g., a truncation relative to a full-length crRNA, e.g., a full-length wild-type crRNA. The overall length of the crRNA can vary and is typically, e.g., 60-120 nucleotides in length, e.g., 60, 70, 80, 90, 100, 110, 120, or any integer within that range, or e.g., 60-90, 60-100, 60-110, 70-120, 80-120, 90-120, or 70-100, 80-100, or 90-100 nucleotides in length.

As used herein, an “editing plasmid” or “donor template” refers to a bacterial plasmid or other nucleic acid construct that comprises a polynucleotide to be integrated using HDR (homology directed recombination) into a bacteriophage genome. The polynucleotide can comprise, e.g., an Acr coding sequence and a sequence to induce a deletion, insertion, or nucleotide substitution in the bacteriophage genome. The plasmid also comprises homology arms, flanking the coding polynucleotide, that comprise homology to a targeted sequence within the bacteriophage genome, i.e., the site of acr gene insertion and genomic modification as described herein. In some embodiments, two distinct homologous regions are present on the template, with each region comprising at least 50, 100, 200, 300, 400, 500, 600, 700, 800, 900 or more nucleotides or more of homology with the corresponding genomic sequence. In some embodiments, the homologous regions correspond to genomic regions that are separated by, e.g., at least 50, 100, 200, 300, 400, 500, 600, 700, 800, 900 bp, or 1, 5, 10, 20, 30, 40, 50, 60, 70, 80, 90, 100 kb or more in the genome. The donor template can be present in any form, e.g., on a plasmid that is introduced into the cell (i.e., an editing plasmid), as a free floating doubled stranded DNA template (e.g., a template that is liberated from a plasmid in the cell), or as single stranded DNA. As the sequence separating the homologous regions on the template will be introduced into the genome by HDR, the present methods can be used to induce precisely defined deletions into the genome (i.e., if the genomic sequence normally present between the homologous regions is absent on the template), to introduce insertions (i.e., if a nucleotide sequence that is not normally present in the genome at the corresponding genomic locus is present on the template between the homologous regions), or to introduce modifications to the genome (i.e., if the nucleotide sequence between the homologous regions on the template differs from the corresponding genomic sequence at one or more nucleotides).

“Anti-CRISPR”, or (Acr) proteins, or (acr) genes, refers to a family of genes and encoded proteins that are associated with inhibition of CRISPR-Cas activity, including RNA-targeting CRISPR-Cas activity such as that provided by Cas13a. Acr proteins effectively against Cas13a can include, e.g., AcrVIA1 and any family members, variants, derivatives, or fragments thereof, polypeptides sharing at least 50%, 60%, 70%, 80%, 90%, 95%, 96%, 97%, 98%, 99% identity to AcrVIA1, or polynucleotides encoding said polypeptides.

### 3. Detailed Description of the Embodiments

Integration of Acr-Encoding Constructs into Phage Genomes

The present disclosure provides novel methods and compositions for generating deletions, insertions, or other genetic modifications in lytic bacteriophages using both Acr-encoding polynucleotides and using RNA-targeting CRISPR-Cas systems such as those comprising Cas13a. In particular embodiments, the present methods and compositions provide a first step in which a polynucleotide encoding an anti-CRISPR protein (Acr) is introduced into a bacteriophage genome. In particular embodiments, the polynucleotide is introduced into the bacteriophage genome by introducing both the bacteriophage (e.g., by infection) and the polynucleotide (e.g., by electroporation) into a bacterial cell, wherein the polynucleotide is present within an editing plasmid in which the coding sequence for the Acr is flanked by homology arms comprising homology to a target site within the bacteriophage genome. It will be appreciated that the introduction of the bacteriophage and the polynucleotide (e.g., the plasmid) into the bacteria can occur in any order, i.e., by introducing the bacteriophage first, by introducing the polynucleotide (e.g., the plasmid) first, or by introducing the bacteriophage and the polynucleotide (e.g., the plasmid) at the same time. By culturing the bacterial cell under conditions conducive to homologous recombination, the polynucleotide is integrated into the bacteriophage genome at the targeted site. In addition, in particular embodiments the editing plasmid (or donor template) also introduces one or more genetic modifications (e.g., gene insertion or deletion) into the bacteriophage genome in proximity to the site of acr gene insertion.

In a subsequent, counter-selection step, bacteriophage isolated from the bacteria, where the phage genome may now include an integrated acr gene if recombination has taken place, are introduced into a second population of bacterial cells comprising an RNA-targeting CRISPR-Cas system. In particular embodiments, the CRISPR-Cas system comprises a Cas13a protein and a crRNA targeting a bacteriophage gene that is essential for its replication. In particular embodiments, the crRNA targets orf120 or orf146 of ΦKZ phage, or an ortholog of either of these coding sequences in another phage. In this way, if recombination has not taken place and the phage genome lacks an integrated acr gene, then the CRISPR-Cas system inhibits the replication of the phage and prevents (or reduces) the formation of lytic plaques. In contrast, if acr gene integration has taken place into the phage genome, then the expressed Acr inhibits the RNA-targeting CRISPR-Cas system and allows bacteriophage replication and, consequently, the formation of plaques. Modified bacteriophage (comprising the integrated acr gene as well as the induced genomic modification) can then be isolated from the plaques and characterized, e.g., using restriction analysis and/or PCR assays, sequencing, or other relevant molecular biological, biochemical, or functional assays.

Accordingly, the present disclosure provides methods and compositions for introducing modifications into a bacteriophage genome comprising the integration of an Acr-encoding gene into the phage genome as well as one or more other modifications such as a deletion, insertion, or nucleotide substitution. The present disclosure also provides bacterial cells for counter selection comprising RNA-targeting CRISPR-Cas systems, for selecting for modified bacteriophage genomes in the counter selection bacterial cells, and for isolating and characterizing successfully modified bacteriophage. The methods and compositions can be used in any bacteriophage (e.g., lytic or temperate) and bacteriophage-infectable bacterial cells, including bacterial cells that do or do not have an endogenous RNA-targeting CRISPR-Cas system.

The methods and compositions described herein can be applied to a number of different bacteriophages. In some embodiments the bacteriophage are jumbo bacteriophage (see, e.g., Yuan and Go, Front Microbiol. 2017; 8: 403). In some embodiments, the bacteriophage are members of the Myoviridae, Podoviridae, or Siphoviridae. Exemplary bacteriophage include, but are not limited to phiKZ, OMKO1, and pamx41.

Any anti-CRISPR can be used in the present methods, so long that the anti-CRISPR inhibits an RNA-guided RNA nuclease such as Cas13 or Cas13a. In particular embodiments, the anti-CRISPR is AcrVIA1, e.g., AcrVIA1 from Listeriophage ΦLS46 (see, e.g., Meeske et al. (2020), the entire disclosure of which is herein incorporated by reference), which is MIYYIKDLKVKGKIFENLMNKEAVEGLITFLKKAEFEIYSRENYSKYNKWFEMWKSPTS SLVFWKNYSFRCHLLFVIEKDGECLGIPASVFESVLQIYLADPFAPDTKELFVEVCNLYE CLADVTVVEHFEAEESAWHKLTHNETEVSKRVYSKDDDELLKYIPEFLDTIATNKKSQK YNQIQGKIQEINKEIATLYESSEDYIFTEYVSNLYRESAKLEQHSKQILKEELN (SEQ ID NO:1) or can be substantially identical to SEQ ID NO:1. A coding sequence for SEQ ID NO:1 is

(SEQ ID NO: 2)
ATGATCTACTATATAAAAGATTTAAAAGTGAAAGGAAAAATATTTGAAA
ATCTAATGAACAAAGAGGCTGTAGAAGGATTAATTACTTTTTTAAAGAA
AGCGGAATTTGAGATATACTCAAGAGAAAATTATTCAAAATACAACAAA
TGGTTTGAAATGTGGAAAAGCCCAACTTCGAGCCTTGTGTTTTGGAAAA
ATTATAGTTTTCGCTGTCATCTTCTTTTTGTCATAGAAAAAGATGGTGA
ATGCCTTGGAATTCCTGCATCTGTTTTTGAATCTGTACTTCAAATTTAC
CTTGCGGATCCGTTCGCTCCCGATACGAAAGAACTTTTTGTTGAGGTTT
GTAATTTATATGAATGTTTAGCGGATGTCACTGTCGTAGAACATTTTGA
AGCGGAAGAATCAGCGTGGCATAAATTAACCCATAATGAGACCGAAGTA
TCAAAAAGAGTCTATAGTAAAGATGATGACGAACTTCTTAAATATATTC
CAGAATTTCTTGACACCATAGCGACAAACAAGAAAAGTCAAAAATACAA
TCAAATTCAAGGAAAAATACAAGAAATTAATAAGGAAATAGCTACACTT
TATGAATCGTCAGAGGATTATATATTTACTGAATATGTTAGTAATTTAT
ATAGAGAGTCTGCAAAGTTGGAGCAACACAGCAAACAAATTTTAAAAGA
GGAGCTAAATTAA

Donor templates comprising polynucleotides encoding the anti-CRISPRs can be generated, e.g., by first amplifying homology arms by PCR from the target locus using the bacteriophage to be modified as a template, and optionally introducing or omitting sequences, as described in more detail elsewhere herein, to induce a modification of the targeted phage genomic locus. A polynucleotide encoding the Acr gene can then also be amplified, and the amplification products can then be introduced into a plasmid, e.g., a pHTERD30T backbone plasmid, and used to transform bacteria such as PAO1, e.g., by electroporation. Transformed cells can be cultured, e.g., in LB with 10 mM MgSO4 and 30 μg/ml gentamicin, and then infected with an appropriate bacteriophage, e.g., wild type ΦKZ phage for PAO1 cells, by adding the phage to the bacterial culture at, e.g., a multiplicity of infection (MOI) of about 1. In some embodiments, the cells are infected prior to introduction of the homologous donor template, and in some embodiments the phage and the template are introduced simultaneously. Following further culture, the cells are lysed, and the phage lysate (which contains both wild-type bacteriophage genomes where recombination has not occurred, and recombinant phage comprising the integrated acr transgene and the genomic modification of interest) is isolated and used to infect a second population of bacterial cells (e.g., PAO1 cells) comprising an RNA-targeting CRISPR-Cas system (e.g., a CRISPR-Cas13a system). The bacteriophage and the CRISPR-Cas system can be introduced in any respective order, i.e., the CRISPR-Cas system first, the bacteriophage first, or the CRISPR-Cas system and bacteriophage at the same time. Any of a number of crRNAs can be used in the CRISPR-Cas system, so long that they target bacteriophage RNA that are essential for bacteriophage replication and, consequently, for the formation of lytic plaques. In particular embodiments, the crRNA targets an essential transcript such as gp120. Plaques that are produced will likely comprise recombinant bacteriophage and can be isolated and characterized, e.g., as described in the present Examples.

In some embodiments, the methods and compositions are used to generate mutations in bacteriophage. In some embodiments, the targeted bacteriophage loci are non-essential, e.g., outside of the coding sequence of an essential gene or encoding a non-essential protein. In some embodiments, the homologous templates are designed such that integration of the acr transgene deletes or otherwise disrupts the targeted locus. In some embodiments, the homologous templates are designed such that the integration of the acr transgene introduces an additional transgene into the targeted locus within the bacteriophage genome. In some embodiments, the homologous templates are designed such that the integration of the acr gene modifies one or more nucleotides within the bacteriophage genome, e.g., to introduce a missense or nonsense mutation into a targeted gene.

In some embodiments, the homologous regions on the template (i.e., homology arms) correspond to genomic phage sequences separated by, e.g., at least about 25, 50, 100, 200, 300, 400, 500, 600, 700, 800, 900 bp or more, or by about 1, 5, 10 kb or more and in some embodiments less than 500, or 1000 bp, and the genomic phage region normally present between the genomic sequences is absent on the template, such that the intervening region between the genomic sequences is deleted from the genome when the acr gene is integrated. In some embodiments, the homologous repair template is used to insert a sequence into the targeted genomic site, e.g., an exogenous sequence is present on the template (in addition to the acr transgene) between the homologous regions that is not normally present at the corresponding genomic locus, such that the exogenous sequence is introduced into the genome when the donor template is integrated into the phage genome by HDR. In some embodiments, the homologous donor template is used to modify the phage genomic sequence at the targeted phage locus, e.g., the nucleotide sequence present on the template (in addition to the acr transgene) between the homologous regions differs from the corresponding genomic sequence at one or more nucleotides, such that the sequence present on the template is introduced into the phage genome during HDR.

### RNA-Targeting CRISPR-Cas Systems

Once recombinant bacteriophage comprising an integrated Acr-encoding polynucleotide and one or more deletions, insertions, or mutations at a targeted locus have been isolated, they are introduced into a second population of bacterial cells comprising an RNA-targeting (or RNA-guided) CRISPR-Cas system. In such methods, a deletion is induced using an RNA-targeting CRISPR-Cas13 system and a crRNA targeting a specific site within the genome, and a homologous repair template is introduced comprising homology to genomic sequence surrounding the targeted site.

The homologous repair template can be present, e.g., on a plasmid, as free-floating DNA (e.g., as liberated from a plasmid in the cell), or as single-stranded DNA, and can be introduced before, at the same time as, or after the infection of the bacteria by the bacteriophage.

The methods and compositions can be used to modify bacteriophage genomes for any purpose, e.g., to delete or modify phage genes, or to introduce exogenous genes into a phage genome, for example for therapeutic purposes to treat bacterial infections, for use as vectors for gene delivery into bacteria, for phage-based vaccination (i.e., as vaccine carriers), for use as bacterial biosensing devices, and for biocontrol in manufacturing.

The present methods and compositions involve the use of bacteria for counter selection that comprise an RNA-targeting CRISPR-Cas system. In particular embodiments, a CRISPR-Cas system is introduced into a cell that does not contain an endogenous system. In particular embodiments, the CRISPR-Cas system comprises a Cas13 protein such as Cas13a (or polynucleotide encoding a Cas13 or Cas13a protein), and a crRNA targeting a bacteriophage transcript that is essential for phage replication in the bacteria. Any RNA-targeting Cas protein, from any source, can be used, so long that a specified RNA sequence is targeted for degradation upon the introduction of a crRNA specific for the targeted sequence. In particular embodiments, Cas13a is used from Leptotrichia, e.g., Leptotrichia shahii.

In some such embodiments, a plasmid or other vector is introduced into a prokaryotic cell containing polynucleotides encoding a Cas13 protein, e.g., Cas13a, operably linked to one or more promoters, such that the Cas13 protein is expressed in the cell. In particular embodiments, the cells are from a P. aeruginosa PAO1 strain harboring the tn7::cas13aLse (SDM084) on the chromosome. In some embodiments, an expression construct is produced and introduced into a plasmid, such that upon introduction of the plasmid into a bacterial cell (such as a Cas13a-expression PAO1 cell) the crRNA is expressed in the cell such that the crRNA and Cas13a can target phage transcripts expressed in the cell. In other embodiments, the Cas13 protein, e.g., Cas13a, is produced in vitro and either introduced directly into the cells or are used to assemble RNPs comprising the Cas13 and a crRNA which are then introduced into the cells using standard methods and as described elsewhere herein.

In some embodiments, a plasmid is introduced into a bacterial cell that includes both a polynucleotide encoding an RNA-targeting Cas13 (i.e., polynucleotide encoding Cas13a), as well as one or more crRNAs that target one or more essential RNA sequences within the bacteriophage genome. In such embodiments, the polynucleotides encoding the crRNA and/or RNA-targeting Cas protein are linked to one or more promoters capable of effecting expression of the crRNA and/or RNA-targeting Cas proteins in the cell, including constitutive and inducible promoters.

crRNAs

The crRNAs used in the present methods and compositions contain a spacer sequence of, e.g., less than 15, 15-20, 20-25, or 25-30 nucleotides in length, e.g., 25 nucleotides, that is complementary to the phage genomic site to be targeted, e.g., a genomic site adjacent to a PAM sequence, as well as one or more repeat sequences that flank the spacer sequences and that comprise sequences that can give rise to stem and loop structures. In wild-type CRISPR-Cas13 systems, the repeat sequences are identical, or virtually identical to one another, although in the present methods modified repeat sequences can also be used, as described in more detail elsewhere herein. Exemplary crRNA target sequences include sequences shown in Table 3, as well as sequences comprising at least about 80%, 85%, 90%, 95%, 96%, 97%, 98%, 99%, or more identity with one or more sequence in Table 3.

Full-length repeat sequences within the crRNAs can be, e.g., from 30-40 nucleotides in length, e.g., 30, 31, 32, 33, 34, 35, 36, 37, 38, 39, or 40 nucleotides, and contain sequences that can give rise to a stem-loop, i.e., where the RNA can fold upon itself and form hybridized base pairs between two complementary regions to form the stem, with the nucleotides located between the two complementary regions and which therefore do not hybridize to form base pairs forming the loop. The stem-loop regions of the present crRNAs can be of any length, e.g., from 10, 11, 12, 13, 14, 15, 16, 17, 18, 19, 20, 21, 22, 23, 24, 25, 26, 27, 28, 29, 30 or more nucleotides, and can contain stems containing 2, 3, 4, 5, 6, 7, 8, 9, 10 or more complementary base pairs. The loops can also be of different lengths, e.g., 2, 3, 4, 5, 6, 7, 8, 9, 10 or more nucleotides. The sequence within the repeat but outside of the stem-loop can also be of various length, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides.

In particular embodiments, a modified crRNA is used, in which one or both of the repeat sequences surrounding a spacer is modified, truncated, or absent so that the two repeats are not identical. In particular embodiments, one or both repeats are modified while still maintaining the stem and loop structures in at least one repeat. In some embodiments, the repeat sequences differ by 1 or more nucleotides, e.g., 1, 2, 3, 4, 5, 6, 7, 8, 9, 10, 11, 12, 13, 14, 15, 16, 17, 18, 19, 20 or more (e.g., 1-15, 1-8, 1-4, 2-6, 3-6) nucleotides. In some embodiments, one of the repeats flanking the spacer is a wild-type or naturally occurring sequence, and the other repeat is a modified sequence. In other embodiments, both of the repeats surrounding a spacer are modified compared to wild-type. In some embodiments, one of the repeats is absent, so that the crRNA comprises (1) a single repeat sequence comprising a stem-loop and (2) a spacer. In some embodiments, one or both repeats is truncated, e.g., from the 5′ or 3′ end of the crRNA, so as to reduce the overall length of the crRNA. In such embodiments, the truncation can remove 1, 2, 3, 4, 5, 6, 7, 8, 9, 10 or more (e.g., 15, 1-8, 1-10, 2-5) nucleotides from the 3′ and/or 5′ end of the crRNA, as compared to a full-length repeat.

In particular embodiments, nucleotides are modified in the stem region and/or the loop region of the repeat. For example, the orientation of base pairs that are formed within the stem region can be reversed, e.g., a G-C base pair could be reversed in one of the stems so that it is C-G in the stem of the other repeat. Such base-pair reversals can be implemented in, e.g., 1, 2, 3, 4, 5 or more base pairs formed within the stem. In particular embodiments, 3 base pairs are reversed, i.e., involving the introduction of 6 nucleotide differences between the two repeat sequences. In certain embodiments, nucleotides within the loop region can be modified. For example, one or more C or G within the loop region can be replaced with an A or T in one of the repeats. Such loop nucleotide changes could be implemented in, e.g., 1, 2, 3, 4 or more (e.g., 1-4, 1-3, 2-4) nucleotides within the loop. In particular embodiments, 3 nucleotides are modified. In some embodiments, repeat nucleotides outside of the stem-loop region are modified so as to differ between the two repeats. In some embodiments, 3 base pairs within the stem region are in reversed orientation in the two repeats, and 3 nucleotides within the loop are different between the repeats, for a total of 9 total differences in the nucleotide sequences of the two repeats

The modification principles described herein for modifying RNA-targeting CRISPR crRNAs, e.g., involving the use of crRNAs with only one repeat sequence, with truncated repeat sequences, or with two repeat sequences containing one or more nucleotide differences, e.g., in the stem, loop, or outside of the stem-loop, can also be used in other CRISPR systems, including other type I systems (e.g., subtypes I-A, I-B, I-U, I-D, I-E, I-F) as well as in type V and type VI systems. As such, in some embodiments, the present disclosure provides a modified crRNA from a type I (e.g., type I-F), type V, or type VI CRISPR system, wherein one or both of the repeat sequences surrounding a spacer is modified, truncated, or absent so that the two repeats are not identical.

### RNA and Protein Preparation

The Cas protein within an RNA-targeting CRISPR-Cas system and/or the crRNA can be prepared using any method. For example, in some embodiments the protein can be purified from naturally occurring sources, synthesized, or more typically can be made by recombinant production in a cell engineered to produce the protein. Exemplary expression systems include various bacterial, yeast, insect, and mammalian expression systems.

The Cas and/or anti-CRISPR polypeptides as described herein can be fused to one or more fusion partners and/or heterologous amino acids to form a fusion protein. Fusion partner sequences can include, but are not limited to, amino acid tags, non-L (e.g., D-) amino acids or other amino acid mimetics to extend in vivo half-life and/or protease resistance, targeting sequences or other sequences. In some embodiments, functional variants or modified forms of the Cas or anti-CRISPR proteins include fusion proteins of an RNA-targeting Cas protein or anti-CRISPR polypeptides and one or more fusion domains. Exemplary fusion domains include, but are not limited to, polyhistidine, Glu-Glu, glutathione S transferase (GST), thioredoxin, protein A, protein G, an immunoglobulin heavy chain constant region (Fc), maltose binding protein (MBP), and/or human serum albumin (HSA). A fusion domain or a fragment thereof may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QLAexpress™ system (Qiagen) useful with (HIS6) fusion partners. As another example, a fusion domain may be selected so as to facilitate detection of the Cas or anti-CRISPR polypeptide. Examples of such detection domains include the various fluorescent proteins (e.g., GFP) as well as “epitope tags,” which are usually short peptide sequences for which a specific antibody is available. Epitope tags for which specific monoclonal antibodies are readily available include FLAG, influenza virus haemagluttinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, an RNA-targeting Cas protein or anti-CRISPR protein is fused with a domain that stabilizes the protein (a “stabilizer” domain). By “stabilizing” is meant anything that increases protein half-life, by any mechanism. Fusions may be constructed such that the heterologous peptide is fused at the amino terminus of an RNA-targeting Cas or anti-CRISPR polypeptide and/or at the carboxyl terminus of an RNA-targeting Cas13 or anti-CRISPR polypeptide.

In some embodiments, the RNA-targeting Cas protein and/or anti-CRISPR polypeptide as described herein comprise at least one non-naturally encoded amino acid. In some embodiments, a polypeptide comprises 1, 2, 3, 4, or more unnatural amino acids. Methods of making and introducing a non-naturally occurring amino acid into a protein are known. See, e.g., U.S. Pat. Nos. 7,083,970; and 7,524,647. The general principles for the production of orthogonal translation systems that are suitable for making proteins that comprise one or more desired unnatural amino acid are known in the art, as are the general methods for producing orthogonal translation systems.

A non-naturally encoded amino acid is typically any structure having any substituent side chain other than one used in the twenty natural amino acids. Because non-naturally encoded amino acids typically differ from the natural amino acids only in the structure of the side chain, the non-naturally encoded amino acids form amide bonds with other amino acids, including but not limited to, natural or non-naturally encoded, in the same manner in which they are formed in naturally occurring polypeptides. However, the non-naturally encoded amino acids have side chain groups that distinguish them from the natural amino acids. For example, R optionally comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amino group, or the like or any combination thereof. Other non-naturally occurring amino acids of interest that may be suitable for use include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analog, glycosylated amino acids such as a sugar substituted serine, other carbohydrate modified amino acids, keto-containing amino acids, amino acids comprising polyethylene glycol or polyether, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

crRNAs can also be prepared, e.g., by chemical synthesis or by in vitro transcription, e.g., using a pUC19 or equivalent vector, e.g., containing a T7 transcription cassette, and purification of the produced crRNAs and, e.g., removal of the 5′ triphosphate group. RNPs, e.g., crRNA-protein complexes comprising the RNA-targeting Cas protein and the crRNA can be prepared by incubating the components together. In particular embodiments, the RNP comprises Cas13a and a crRNA. Methods of chemically synthesizing RNA or of producing RNA in in vitro transcription systems are well known in the art.

The efficacy of crRNAs, RNA-targeting CRISPR-Cas systems, and RNPs can be assessed using any of a number of assays. In particular embodiments, the ability of an RNA-targeting CRISPR-Cas system to prevent bacteriophage replication, and/or the ability of an anti-CRISPR to rescue bacteriophage replication, can be assessed by titering phage comprising a polynucleotide encoding the anti-CRISPR onto bacteria comprising the RNA-targeting CRISPR-Cas system, and detecting the formation of lytic plaques. The generation of deletions, insertions, and modifications in phage genomes can also be assessed by, e.g., delays or other alterations in the growth of infected cells, as well as by standard molecular biology or biochemical methods for detecting deletions, insertions, or genomic modifications such as PCR, Sanger sequencing, whole genome sequencing, or Southern Blotting.

Delivery into Cells

Introduction of the RNA-targeting CRISPR-Cas system polynucleotides, polypeptides, RNPs, and/or of anti-CRISPRs (e.g., donor templates or editing plasmids), into bacterial cells can take different forms. For example, in some embodiments, the polypeptides and/or RNPs themselves are introduced into the cells. Any method for the introduction of polypeptides or RNPs into cells can be used. For example, in some embodiments, electroporation, bacteriophage-mediated, or liposomal or nanoparticle delivery to the cells can be employed. In other embodiments, one or more polynucleotides encoding a crRNA and/or RNA-targeting Cas (such as Cas13a), or encoding an anti-CRISPR, are introduced into a cell and the crRNA and/or RNA-targeting Cas protein, and/or the anti-CRISPR protein, are subsequently expressed in the cell. In some embodiments, the polynucleotide is an RNA molecule. In some embodiments, the polynucleotide is a DNA molecule.

In some embodiments, the crRNA, Cas protein, and/or anti-CRISPR are expressed in the cell from RNA encoded by an expression cassette, wherein the expression cassette comprises a promoter operably linked to a polynucleotide encoding the crRNA, Cas protein, and/or anti-CRISPR protein. In some embodiments, the promoter is heterologous to the polynucleotide encoding the crRNA, anti-CRISPR and/or Cas protein. In some embodiments, promoters are inducible or repressible, such that expression of a nucleic acid operably linked to the promoter can be expressed under selected conditions.

In embodiments where a polynucleotide is introduced that encodes an appropriate crRNA or polynucleotide encoding a Cas protein or an anti-CRISPR, any suitable promoter can be used that will lead to a level of expression that is higher than the level in the absence of the construct. Any level of expression that is sufficient to, e.g., increase or decrease bacteriophage replication as described herein can be used.

In some embodiments, polynucleotides, e.g., homologous repair template encoding an anti-CRISPR, or polynucleotide encoding a crRNA and/or RNA-targeting Cas protein, are introduced into bacteria using phage, e.g., a phage delivery vector comprised of ssDNA or dsDNA that delivers DNA cargo to target cells. Any phage capable of introducing a polynucleotide into the target cell can be used. The phage could be, e.g., a tailed phage or a filamentous phage, that carries an entirely designed genome or that has heterologous genes introduced into an otherwise natural genome.

In other embodiments, polynucleotides, e.g., homologous repair template encoding an anti-CRISPR, or polynucleotide encoding a crRNA and/or RNA-targeting Cas protein, are introduced into bacteria using bacterial conjugation. In some embodiments, polynucleotides are introduced into target prokaryotes using E. coli as a conjugative donor strain, e.g., using mobilizable plasmids that transfer their genetic material, e.g., homologous repair template encoding an anti-CRISPR, or polynucleotide encoding a crRNA and/or RNA-targeting Cas protein.

In certain embodiments, the crRNA and/or Cas protein are produced in vitro and introduced directly into cells, either individually or as a pre-formed RNP, i.e., a crRNA-Cas protein complex.

In certain embodiments, the crRNA and Cas protein are introduced into the cell by directly introducing RNA into the cell, e.g., crRNA and/or mRNA encoding an RNA-directed Cas protein such as Cas13a.

In some embodiments, the crRNAs, anti-CRISPR, and/or RNA-targeting CRISPR-Cas system components are introduced into cells using modified RNA. Various modifications of RNA are known in the art to enhance, e.g., the translation, potency and/or stability of RNA when introduced into cells. In particular embodiments, modified mRNA (mmRNA) is used, e.g., mmRNA encoding an RNA-targeting CRISPR-Cas system component or anti-CRISPR. In other embodiments, modified RNA comprising a crRNA is used. Non-limiting examples of RNA modifications that can be used include anti-reverse-cap analogs (ARCA), polyA tails of, e.g., 100-250 nucleotides in length, replacement of AU-rich sequences in the 3′UTR with sequences from known stable mRNAs, and the inclusion of modified nucleosides and structures such as pseudouridine, e.g., N1-methylpseudouridine, 2-thiouridine, 4′thioRNA, 5-methylcytidine, 6-methyladenosine, amide 3 linkages, thioate linkages, inosine, 2′-deoxyribonucleotides, 5-Bromo-uridine and 2′-O-methylated nucleosides. A non-limiting list of chemical modifications that can be used can be found, e.g., in the online database crdd.osdd.net/servers/simamod/.

Any prokaryotic cell that can be infected by a bacteriophage can be used in the present methods. Exemplary prokaryotic cells can include but are not limited to, those used for biotechnological purposes, the production of desired metabolites, E. coli and human pathogens. Examples of such prokaryotic cells can include, for example, Escherichia coli, Pseudomonas sp., Corynebacterium sp., Bacillus subtilis, Streptococcus pneumonia, Pseudomonas aeruginosa, Staphylococcus aureus, Campylobacter jejuni, Francisella novicida, Corynebacterium diphtheria, Enterococcus sp., Listeria monocytogenes, Mycoplasma gallisepticum, Streptococcus sp., or Treponema denticola. In some embodiments, prokaryotic cells include pathogenic cells and/or antibiotic resistant cells.

### Kits

Other embodiments of the compositions described herein are kits comprising one or more crRNAs, RNA-targeting CRISPR-Cas protein or proteins, homologous repair templates, polynucleotides encoding one or more crRNAs of the invention and/or encoding one or more RNA-targeting Cas protein or proteins (such as Cas13a) or an anti-CRISPR (such as AcrVIA1), and/or RNPs comprising crRNAs and RNA-targeting Cas proteins. The kit typically contains containers, which may be formed from a variety of materials such as glass or plastic, and can include for example, bottles, vials, syringes, and test tubes. A label typically accompanies the kit, and includes any writing or recorded material, which may be electronic or computer readable form providing instructions or other information for use of the kit contents.

In some embodiments, the kits can further comprise instructional materials containing directions (i.e., protocols) for the practice of the methods of this invention (e.g., instructions for using the kit for inducing deletions, insertions, or other modifications into bacteriophage genomes. While the instructional materials typically comprise written or printed materials, they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD-ROM), and the like. Such media may include addresses to internet sites that provide such instructional materials.

### 4. Examples

Bacteriophages (or phages) possess many genes of unknown function and thus genetic tools are required to understand their biology and to enhance their antimicrobial efficacy. Pseudomonas aeruginosa jumbophage ΦKZ and its relatives are a broad host range phage family that assemble a proteinaceous “phage nucleus” structure during infection. Due to the phage nucleus, DNA-targeting CRISPR-Cas is ineffective against this phage and thus there are currently no reverse genetic tools for this family. Here, we develop a DNA phage genome editing technology using the RNA-targeting CRISPR-Cas13a enzyme as a selection tool, an anti-CRISPR gene (acrVIA1) as a selectable marker, and homologous recombination. Precise insertion of foreign genes, gene deletions, and addition of chromosomal fluorescent tags into the ΦKZ genome were achieved. Deletion of orf39, which encodes a tubulin-like protein (PhuZ) that centers the phage nucleus during infection, led to the mispositioning of the phage nucleus but surprisingly has no impact on phage replication, despite a proposed role in capsid trafficking. An endogenous fluorescent tag placed on gp93, a proposed “inner body” protein in the phage head revealed a protein that is injected with the phage genome, localizes with the maturing phage nucleus, and then is massively synthesized around the phage nucleus during phage maturation. Successful editing of other phages that resist DNA-targeting CRISPR-Cas systems [e.g., OMKO1, PaMx41] demonstrates the flexibility of this method. The data suggested that the RNA-targeting Cas13a system holds great promise for becoming a universal genetic editing tool for intractable phages. This phage genetic engineering platform enables the systematic study of phage genes with unknown function and the precise modification of phages for use in a variety of applications.

### Introduction

Here, we develop the CRISPR-Cas13a system as a novel genetic engineering approach for ΦKZ. Using Cas13a targeting an essential transcript, we select for phage DNA that has undergone homologous recombination resulting in a desired genetic change along with the acquisition of an anti-Cas13a trans gene, acrVIA1 (derived from Listeriophage ΦLS46 [23]), as a selectable marker. This approach allows us to precisely insert foreign gene fragments into the ΦKZ genome, knock out non-essential genes, and fuse fluorescent tags to individual genes. Importantly, the same guide can be used for any genomic manipulation for the same phage strain as engineered phages are identified based on the acquisition of the Cas13a inhibitor, not a change in the target sequence. Our work establishes a Cas13a-based phage engineering strategy that could be a universally powerful tool for engineering phages that are intractable with DNA-targeting genetic engineering techniques.

### Results

### Optimization of CRISPR-Cas13a for Efficient Phage Targeting

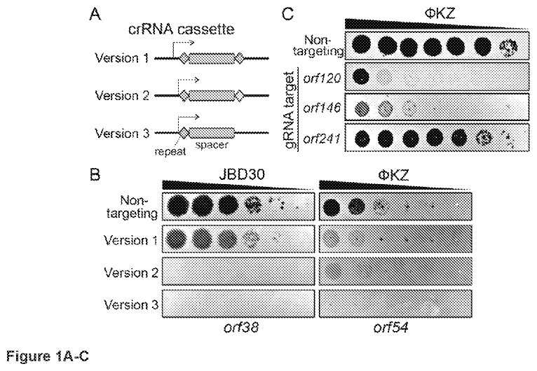

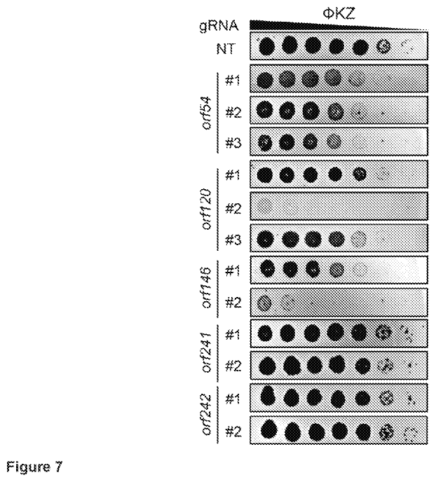

Cas13a is an RNA-guided RNA nuclease that can block ΦKZ replication in P. aeruginosa PAO1. We previously targeted ΦKZ by expressing crRNA guides from a plasmid (FIG. 1A, Version 1) [19]. For the effective elimination of WT phages in the population, we first sought to enhance activity of crRNA guides. We designed Version 2 (V2) with the repeat-spacer-repeat unit moved to the +1 transcription start site and the second direct repeat (DR) mutated to remove repeat homology (FIG. 1A). To further simplify the crRNA cassette, we omitted the second DR and generated V3 (FIG. 1A). Using the same spacer, both V2 and V3 provided more robust defense against phage JBD30 and ΦKZ compared with V1 (FIG. 1A). For simplicity, we selected the V3 cassette that has a single repeat to express crRNAs against ΦKZ. We designed a number of spacer sequences to target various ΦKZ gene transcripts. Strong targeting was observed for some crRNAs, to the point that escaper phages could be isolated, such as the two spacers matching orf120 and orf146 transcripts, but not all crRNAs were efficacious (FIGS. 1B and 7). Given the variability of targeting, for the remainder of this report, we use orf120-targeting gRNA #2 as our primary guide to screen for engineered phages. We refer the PAO1 strain simultaneously expressing Cas13a and orf120-gRNA #2 to as Cas13a counter-selection strain. We describe below how the same guide can be used to facilitate the engineering of distinct genomic loci.

### Screening of ΦKZ Recombinants by CRISPR-Cas13a Counter Selection

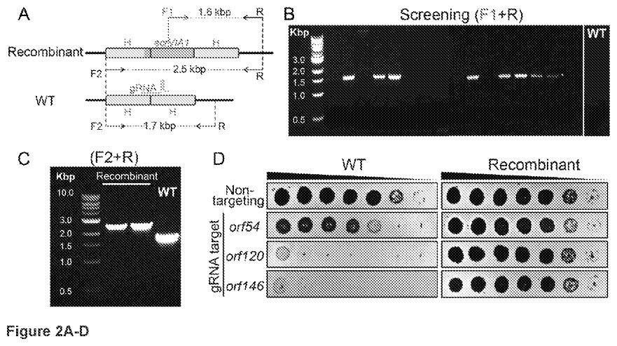

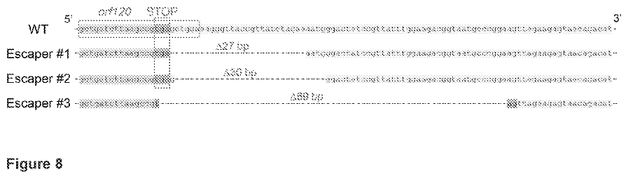

To avoid disrupting any essential genes that are required for phage replication, we first attempted to insert acrVIA1 immediately downstream of ΦKZ major capsid gene (orf120). A template DNA substrate for homologous recombination, composed of ˜600-bp homology arms flanking acrVIA1 was cloned into a plasmid, referred to as an editing plasmid (FIG. 2A). After infecting a PAO1 strain with this plasmid to allow recombination, the phage lysate was then titrated on the lawn of the Cas13a counter-selection strain to eliminate WT phages. To screen for recombinants, individual plaques were examined for acrVIA1 integration via PCR, showing that 8 out of the 16 tested phage plaques generated the expected 1.6 kbp band, which was not detectable in a WT plaque (FIG. 2B). Amplification of the entire region also revealed the expected size increase, from ˜1.7 kbp of WT to ˜2.5 kbp of recombinants, and sequencing confirmed the correct integration junction (FIG. 2C). Recombinant phages propagated well on bacteria expressing crRNAs targeting other genomic loci due to the expression of acrVIA1, which abolishes Cas13a immunity regardless of the crRNA sequence (FIG. 2D). Three randomly selected phages that escaped Cas13a targeting but screened negative for the acrVIA1 integration contained genomic deletions ranging from 27 bp to 69 bp starting immediately downstream of the orf120 stop codon, disrupting the protospacer (FIG. 8). While the crRNA used is therefore not inescapable, the recombination efficiency to insert the selectable marker is clearly efficient enough to enable facile identification of the desired mutants.

To test the flexibility of this nascent genetic technology and generate new biological insights of phage ΦKZ, we next knocked out (or attempted to knock out) multiple genes; phuZ (orf39), orf54, orf89-orf93, orf93, orf146, orf241, and orf241-orf242 (summarized in Table 1), in addition to attempting to add chromosomal fluorescent tags onto orf54 and orf93. The successes, failures, and next steps are discussed below, along with the new insights gained. Whole genome sequencing of two deletion mutants, ΔphuZ and Δorf93, revealed no other mutations. The accuracy of this system highlights an important advantage of adapting an RNA-targeting system CRISPR-Cas13a to select for edits in phage genomes, compared to direct DNA cleavage.

### Characterization of PhuZ and Gp93 Using Engineered ΦKZ Mutants

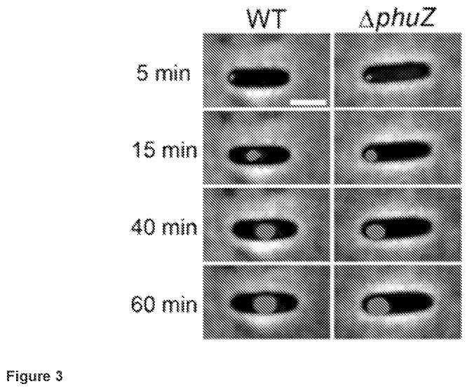

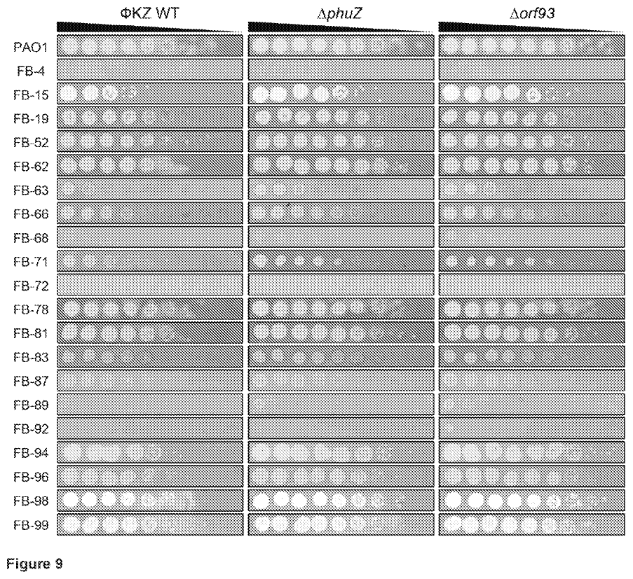

PhuZ (gp39) is conserved across jumbo phages and some megaphages [25]. It constitutes a bipolar spindle to center the phage nucleus during phage intracellular development [26, 27], and “treadmill” newly synthesized phage capsids from the cell inner membrane to the phage nucleus for DNA packaging [28]. These functions made us speculate that PhuZ might be essential or important for phage growth, however, this is not the case. ΔphuZ mutants exhibited a similar burst size (24 phage particles per infected bacterial cell vs. 19 of WT phage) under our experimental conditions. Under the microscope, we observed that the phage nucleus was mispositioned in cells infected by ΔphuZ mutants, in contrast to WT infections (FIG. 3). Subcellular location of the phage nucleus in cells infected with ΔphuZ mutants showed a wide distribution compared with cells infected with WT phages or ΔphuZ mutants complemented with PhuZ in trans, where ˜80% of phage nuclei were centered. This is consistent with the previous findings that trans over-expression of catalytic mutant PhuZ resulted in mispositioning of the phage nucleus [27, 29]. ˜25% of mutant-infected cells still positioned the phage nucleus at the cell center, a phenotype most commonly seen in shorter cells (Pearson correlation coefficient=0.486, p<0.001), in contrast with WT infection (Pearson correlation coefficient=0.029, p=0.505). Considering that PhuZ is proposed to traffic phage capsids from the cell inner membrane to the phage nucleus [28, 30], we speculate that this is only required during specific conditions or not at all. Taken together, these data suggested that PhuZ positions the phage nucleus at the cell center during infection, but the removal of PhuZ does not have a significant impact on ΦKZ growth in laboratory conditions. Efficiency of plaquing of WT and ΔphuZ on P. aeruginosa clinical strains is presented in FIG. 9.

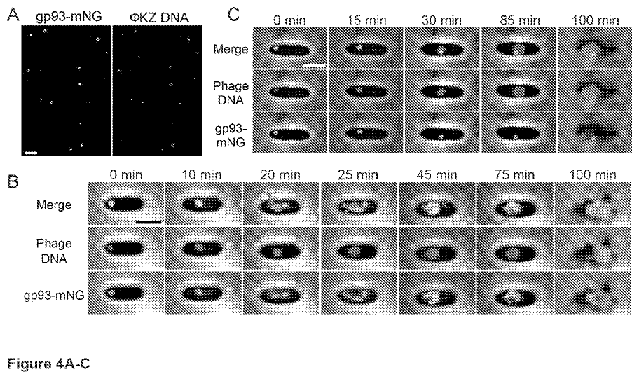

orf93 encodes gp93, a high copy number “inner body (IB)” protein that is packaged in the phage head [31, 32]. Deletion of orf93 also yielded viable phage with no obvious growth defect. We next analyzed the inserted fluorescent chromosomal label at the C-terminus of the protein, which is notable as the first endogenous protein tag in this phage family. Labeled gp93 was observed in the mature virion (FIG. 4A), as expected from previous mass spectrometry studies [32] and is clearly injected during phage infection. Time-lapse movies revealed the fluorescently labeled protein as a green focus representing an infecting phage initially colocalized with phage DNA stained by DAPI (blue) at the cell pole (FIG. 4B). Over time, the green focus translocated from the cell pole to the cell center and remained bound to the phage nucleus. As new gp93-mNeonGreen was expressed from the phage genome, more and more green signals concentrated on the surface of the phage nucleus, while some foci appeared on the cell inner membrane. Finally, cells lysed and released fluorescent phage progeny. To confirm that the protein that appears to be injected was not rapidly synthesized de novo, we monitored the infection behaviors of WT phages loaded with gp93-mNeonGreen expressed from a plasmid during phage production, but where no new fluorescent protein could be made during infection (FIG. 4C). Similar to the engineered phage, the green focus migrated from the cell pole to the cell center along with phage DNA on the surface of the phage nucleus until cell lysis. Therefore, the IB protein gp93 is not only packaged in the phage head, but is also injected with the phage genome, co-localizes with the nascent nucleus, trafficks with the mature nucleus, and is massively synthesized late during infection. The ability to endogenously label phage proteins, as demonstrated here, will be beneficial for characterizing ΦKZ virion and cell biology in the future.

To assess whether the deletions of phuZ or orf93 impact growth in a strain-dependent manner, we challenged a panel of 21 P. aeruginosa clinical strains with ΔphuZ and Δorf93 mutants to investigate whether these genes are dispensable for ΦKZ replication in PAO1 but not elsewhere. Plaque assays showed that host ranges of both mutants were similar, if not identical, to WT (FIG. 9), suggesting that these knockouts, and Cas13a-mediated genetic engineering in general, has no impact on the ΦKZ host range.

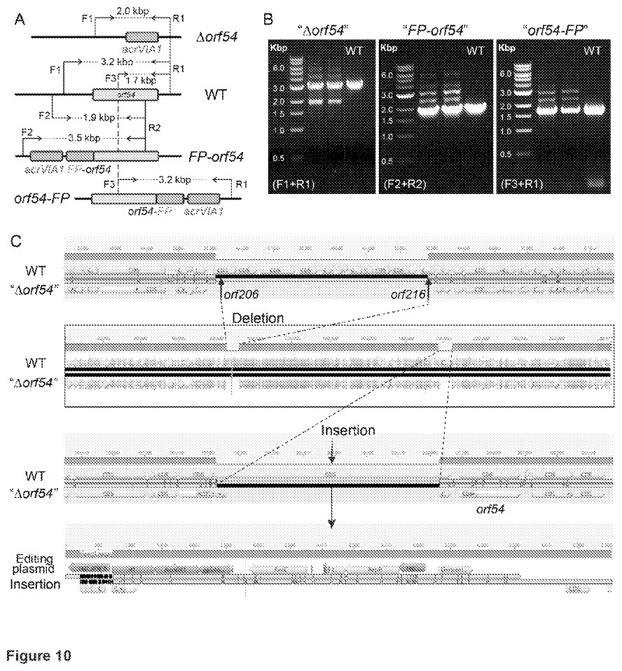

The phage nucleus is primarily composed of gp54 [27]. We were unable to knockout or fluorescently label orf54, even when wild-type gp54 was provided by expressing from a plasmid in trans. The primers used to amplify the region of editing generated multiple bands for both deletion and tag-addition mutant variants (FIG. 10A). N- or C-terminal fusion of gp54 with mCherry tags yielded the same fate. Whole genome sequencing of an isolated orf54 “pseudo knock-out” strain revealed that part of the editing plasmid was integrated upstream of orf54, while the orf54 gene was left intact (FIG. 10B). A similar attempt to delete the structural gene (orf146) and a cluster of IB genes (orf89-orf93) also failed, while deletion of accessory genes orf241 and orf241-242 succeeded but yielded no obvious phenotype. These results highlighted that the CRISPR-Cas13a counter selection system is a strong and efficient phage genome engineering tool, but the modification of phage essential genes remains challenging.

### Precise Genome Engineering of Clinical Phage OMKO1

We next explored the versatility of our phage engineering platform by editing the genome of a clinical jumbo phage. We selected OMKO1, a P. aeruginosa phage with an approximate 280 kbp genome that has 90.1% nucleotide sequence identity to ΦKZ. OMKO1 is a potentially ideally therapeutic phage since P. aeruginosa strains that evolve resistance to infection become sensitized to small-molecule antibiotics. This phage has been used for phage therapy as emergency treatment for chronic infections caused by antibiotic-resistant P. aeruginosa38 and it is currently being tested in a phase I/II clinical trial (CYstic Fibrosis bacterioPHage Study at Yale, https://clinicaltrials.gov/ct2/show/NCT04684641). With this phage, we tested whether we could insert ‘DNA barcodes’ without impacting host range for downstream clinical applications. Insertion of a DNA tracking signature into clinical phages would enable differentiation from naturally occurring phages during the manufacturing process and after administration to patients.

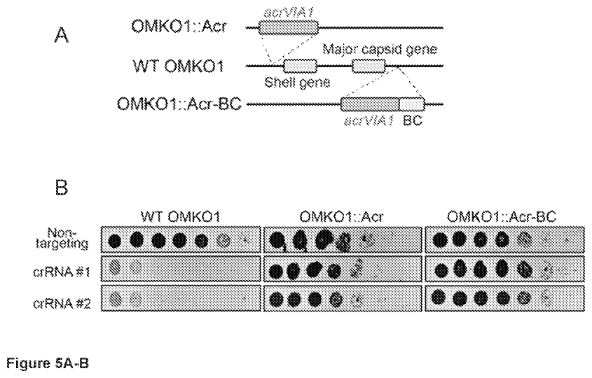

Two engineered OMKO1 strains were generated, one with acrVIA1 and a 120 nucleotide barcode inserted downstream of the capsid gene, and another with acrVIA1 integrated upstream of the shell gene (orf54 homologue; FIG. 5A and Table 1). The presence of the desired inserts was confirmed with WGS of both OMKO1 engineered strains; no unintended genetic changes occurred. Moreover, both strains exhibited strong resistance to Cas13a targeting (FIG. 5B), owing to the expression of acrVIA1 from the phage genomes. The host range and virulence of the two engineered OMKO1 variants together with the parental phage was then assessed on 22 P. aeruginosa clinical strains (including PAO1). The experiment was performed in a microplate liquid assay, where phage variants were individually mixed with each host strain at a multiplicity of infection (MOI) of approximately 1 and 0.01. All three phages displayed the same host range (Table 5) and were capable of infecting and suppressing the growth of 20 out of 22 (91%) clinical strains tested. Infections at high MOI (MOI=1) resulted in a broader host range and greater bacterial growth suppression, while low MOI (MOI=0.01) infections suppressed the cell growth of 12 out of 22 (55%) hosts. All phages exhibited similar virulence across all hosts with small differences in 5 out of 22 strains (marked with an asterisk in Table 5). Altogether, these results indicate that the OMKO1's host range was not affected and virulence was impacted only modestly by inserting acrVIA1 or acrVIA1 and a barcode in the two selected genome locations under the tested conditions.

### Application of CRISPR-Cas13a Phage Engineering to Another Virulent Phage

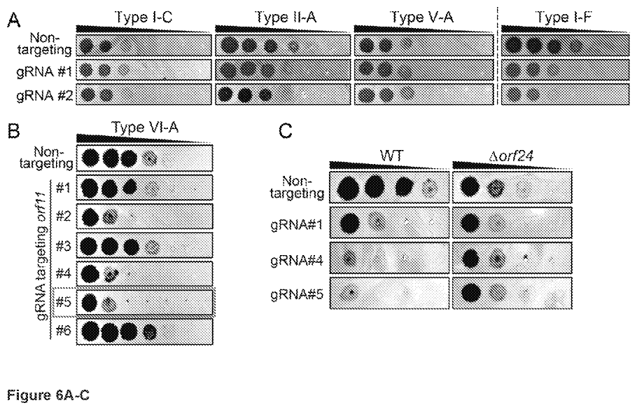

To evaluate the applicability of the CRISPR-Cas13a-mediated genome editing approach to other virulent phages, we selected P. aeruginosa phage PaMx41. PaMx41 was isolated from environmental and sewage water samples in Central Mexico, and belongs to the Podoviridae family [34]. Its genome is approximately 43.5 kbp long and harbors 55 open reading frames (ORFs), ˜70% of which have unknown functions [35]. Remarkably, PaMx41 appeared to be resistant to many DNA-targeting CRISPR-Cas systems (Type I—C, II-A, and V-A) and showed partial sensitivity (˜10-fold reduction in efficiency of plating) to Type I-F to a degree that is not sufficient for counter selection (FIG. 6A). In contrast, when the transcripts of the major capsid gene (orf11) were targeted by CRISPR-Cas13a, PaMx41 exhibited strong sensitivities to specific crRNAs (FIG. 6B). Following the same approach as we developed to engineer ΦKZ, we successfully replaced one hypothetical gene (orf24) and its downstream non-coding region with acrVIA1 and isolated a pure mutant strain using an efficient gRNA (#5) (FIG. 6B, Table 1). The mutant showed expected anti-CRISPR activities against different orf11-targeting crRNAs, indicating that the incorporated acrVIA1 was expressed and properly functioning (FIG. 6C). Notably, initial PCR screening for recombinant plaques showed that 100% of surviving phages were desired recombinants, with no spontaneous escaper plaques. The data suggested that the RNA-targeting Cas13a system holds great promise for becoming a universal genetic editing tool to deal with previously intractable phages.

### DISCUSSION

We exploited the RNA-targeting CRISPR-Cas13a system in conjunction with homologous recombination to achieve genetic modification of jumbo phage ΦKZ and phage PaMx41 that all resist DNA-targeting CRISPR-Cas systems. CRISPR-Cas13a-mediated counter-selection recovered rare (10⁻⁵) phage recombinants from a large pool of wild-type phages. Many studies have uncovered that phages can hamper CRISPR-Cas activities, for example, by repressing transcription of CRISPR-Cas components [36, 37], possessing covalent DNA modifications [38-41], or encoding anti-CRISPR proteins (recently reviewed in [42]). Furthermore, the assembly of a proteinaceous nucleus-like structure that shields phage genomes from attack by distinct DNA-targeting nucleases [19, 20] represents the ultimate “anti-CRISPR/anti-RM” mechanism. Therefore, development of a phage genomic manipulation approaches that target a relatively constant and exposed molecule, mRNA, with Cas13a may provide a near-universal approach. Moreover, Cas13 is rarely encoded in bacteria and thus most phages are not expected to encode anti-Cas13a proteins.

One major challenge of using Cas13a in our experience has been the wide variability of crRNA efficacy, which is likely due to numerous factors. Future studies focusing on the optimization of crRNA design or perhaps the implementation of other Cas13 enzymes or Cas7-11 [43, 44] will be important. However, we circumvent this problem by implementing an anti-CRISPR selectable marker [45] to ensure that the same strong guide can be used for all genetic manipulations. The downside is that this limits the user to a single perturbation, however, double and triple mutants are possible in principle if one designs and optimizes crRNAs to target the specific sites that await editing and instead of inserting an acr marker, alters the protospacer sequence during manipulation.

Deletion of a specific phage gene allows us to study its biological function. Here, we observed that ΔphuZ mutant mispositioned the phage nucleus during viral intracellular development. Previous studies revealed that newly assembled phage capsids trafficked along PhuZ filaments towards the phage nucleus for viral DNA packaging [28]. However, our work suggests that capsid translocation is not dependent on PhuZ. Moreover, loss of PhuZ appeared not to affect burst size, in contrast with a previous report using over-expression of a catalytic mutant and microscopy to estimate burst size [29]. Overall, phuZ seems to be a bona fide nonessential gene for ΦKZ. The evolutionary advantage of encoding this tubulin in this jumbophage and many others requires further investigation. Furthermore, an endogenous fluorescent label on gp93 demonstrated that it is packaged in the phage head, ejected with the genome, and massively synthesized later in infection, with peri-nuclear localization. The labeling not only allows us to visualize individual virions under the microscope, but also to observe the injection of this inner body protein into the host cell, which had been previously proposed with little evidence [32, 46].

Altogether, the RNA-targeting CRISPR-Cas13a counter-selection tool should be applicable to a broad range of phages and enable downstream high throughput genetic approaches for phage engineering. The ability to precisely and efficiently generate synthetic phages with desired features will not only benefit phage therapeutic applications but also advance our understanding of fundamental phage biology and phage-bacteria interactions.

### Materials and Methods

### Strains, DNA Oligonucleotides and Plasmid Constructions

All bacterial and phage strains, spacer sequences, and primers used in this study are listed in Tables 1-5.

The crRNAs designed for CRISPR-Cas13 targeting were constructed in the pHERD30T backbone. The pHERD30T-crRNA Version 2 was constructed by thermal annealing of oligonucleotides oSDM465 and oSDM466 and phosphorylation by polynucleotide kinase (PNK). The annealed product was introduced by Gibson assembly into pHERD30T linearized by PCR using oligonucleotides oSDM457 and oSDM458. Proper construction of the expression vector was verified by Sanger sequencing. The pHERD30T-crRNA Version 3 was constructed just as for Version 2, but the crRNA-coding insert was instead composed of oligonucleotides oSDM455 and oSDM456. Both Version 2 and Version 3 of this plasmid were designed such that cleavage by BsaI would generate a linear plasmid that would accept annealed oligonucleotide spacers via ligation. Oligonucleotide pairs with repeat-specific overhangs encoding spacer sequences were annealed and phosphorylated using T4 polynucleotide kinase and then cloned into the BsaI-digested empty vectors. Cloning procedures were performed in commercial E. coli DH5a cells (New England Biolabs) according to the manufacturer's protocols. The resulting crRNA plasmids were electroporated into P. aeruginosa PAO1 strain harboring the tn7::cas13aLse (SDM084) on the chromosome as described previously [19]. Gene expression was induced by the addition of L-arabinose at a final concentration of 0.3% and isopropyl β-D-1-thiogalactopyranoside (IPTG) at a final concentration of 1 mM.

To construct template plasmids for homologous recombination, homology arms of >500 bp in length were amplified by PCR using the ΦKZ genomic DNA as the template. To prevent Cas13a cleavage, several synonymous mutations were introduced into the crRNA-targeting site of the left orf120 homology arm by designing the reverse primer (JG064) to contain appropriate mismatches. The acrVIA1 gene was amplified from plasmid pAM383 [23], a gift from Luciano Marraffini, The Rockefeller University. PCR products were purified and assembled as a recombineering substrate and then inserted into the NheI site of the pHERD30T backbone by Gibson Assembly (New England Biolabs) following the manufacturer's protocols. The resulting plasmids were transformed into PAO1 by electroporation.

### Isolation of Phage Recombinants

Host strains bearing recombination plasmids were grown in LB supplemented with 10 mM MgSO4 and 50 μg/ml gentamicin, at 37° C. with aeration at 250 rpm. When OD600 is around 2, Wild type ΦKZ was added into the culture at a MOI (multiplicity of infection) of 1 to allow infection to occur for ˜18 hours. 2% volume of chloroform was added into the infection culture and left to shake gently on an orbital shaker at room temperature for 15 min, followed by centrifugation at 4,000×g for 15 min to remove cell debris. The supernatant lysate was further treated with 2% of chloroform for 15 min and centrifuged again under the same conditions, followed by a 30-min treatment with DNase I (New England Biolabs) at 37° C. The resulting phage lysate containing both WT phages and recombinants are tittered on PAO1 strains bearing the CRISPR-Cas13a system with the most efficient crRNA (orf120 guide #2) to screen for recombinants. Individual phage plaques were picked from top agar and purified for three rounds using the CRISPR counter-selection strain to ensure thorough removal of any remaining WT or escapers. Whether or not they are recombinant phages or Cas13a escaper phages were determined by PCR using appropriate pairs of primers amplifying the modified regions of the phage genome. Identified phages were further confirmed and analyzed by sequencing the PCR products or the whole genomes and then stored at 4° C.

### Phage Plaque Assay

Host strains were grown in LBM (LB supplemented with 10 mM MgSO4), 50 μg/ml gentamicin, 1 mM IPTG and 0.3% arabinose inducers for gene expression, at 37° C. with aeration at 250 rpm for overnight. Phage spotting assays were performed using 1.5% LB agar plates and 0.42% LB top agar, both of which contained 10 mM MgSO4 and inducers. 100 μl of appropriate overnight culture was suspended in 3.5 ml of molten top agar and then poured onto an LB+10 mM MgSO4 agar plate, leading to the growth of a bacterial lawn. After 10-15 min at room temperature, 2 μl of ten-fold serial dilutions of phages was spotted onto the solidified top agar. Plates were incubated overnight at 37° C. Plate images were obtained using Gel Doc EZ Gel Documentation System (BioRad) and Image Lab (BioRad) software.

### Microplate Liquid Assay

Fresh overnight cultures were diluted to a cell concentration of 1×108 cfu/ml in TSB media supplemented with 10 mM MgSO4. Phage lysates were added to reach a MOI of ˜1 and ˜0.01 in a Corning Costar 96-well clear flat-bottom microplate (Thermo Fisher Scientific) sealed with a Breathe-Easy® sealing membrane (Merck KGaA). After the infection cultures were incubated at room temperature for 20 min, plates were incubated at 37° C., 800 rpm for 8 hours in a BioTek LogPhase 600 plate reader (Agilent Technologies, Inc.). Cell growth was monitored by measuring OD600 every 20 min. Each phage-host combination was performed in three biological replicates.

### Data Analysis

Growth curves for each phage-host combination were obtained by plotting OD600 after blank correction (baseline adjustment) against time. Each growth curve was transformed into a single numerical value by calculating the area under the curve (AUC) using the Trapezoid method. Then, AUCs were normalized as a percentage of the AUC of their corresponding uninfected control following the equation,

$$Liquid assay score = \frac{\begin{matrix}A U C ( positive control ) - \\ A U C ( phage treatment )\end{matrix}}{A U C ( positive control )} \times 100$$

The resulting value, defined as the “liquid assay score”, represents how well the phage strain can repress the growth of a bacterial population over the course of the 8-hour experiment. No inhibition of bacterial growth would result in a liquid assay score of 0, and complete suppression would translate into a score of 100. Liquid assay scores were averaged using data from three biological replicates.

### Burst Size Measurement

Phage burst size was determined by one-step growth curve experiments. Briefly, the host PAO1 strain was grown in LBM to OD600 ˜0.4. 1 ml of the cell culture was then centrifuged at 4,000×g for 2 min and the cell pellet was resuspended in 50 μl of fresh LBM. Appropriate amount of phages was mixed with the cell culture to achieve an MOI of 0.01 to limit to single infections. The mixture was incubated on ice for 20 min for phage adsorption and transferred to a 37° C. heat block for 10 min to trigger phage DNA injection. The infection mixture was centrifuged at 10,000×g for 2 min. Transfer 10 μl of the supernatant into 990 μl of ice-cold SM buffer supplied with 2% chloroform. Titer to calculate the number of free phages. After discarding the supernatant to remove free phage particles, the pellet was resuspended in 1 ml of LBM, followed by incubation at 37° C. with shaking at 250 rpm. Samples were collected at 10-min intervals until 90 min, and phage titer was determined immediately. Phage burst sizes were calculated by dividing the phage titers at ˜50 min by the initial phage titers after subtracting free phages.

### Single-Cell Infection Assay

1 ml of host cells was grown in LBM (LB supplemented with 10 mM MgSO4) and 50 μg/ml gentamicin (if necessary), at 37° C. with aeration at 250 rpm for overnight. The overnight culture was diluted 1:100 into 5 ml of LBM and grown at 37° C. with 250 rpm shaking until OD600 ˜0.4. Next, 1 ml of cell culture was collected by centrifugation at 3,000×g for 2 min at room temperature and concentrated by 25-fold in fresh LBM. 10 μl of cells were then mixed with 10 μl of appropriate phage strains to reach an appropriate MOI, followed by incubation at 30° C. for 10 min to allow for phage infection. The infection mixture was further diluted by 10-fold into 50 μl of fresh LBM at room temperature. 1 μl of the diluted culture was gently placed onto a piece of agarose pad (˜1 mm thick) with 1:5 diluted LBM, arabinose (0.8%), and DAPI (5 μg/ml; Invitrogen& #153, No. D1306). A coverslip (No. 1.5, Fisher Scientific) was gently laid over the agarose pad and the sample was imaged under the fluorescence microscope at 30° C. within a cage incubator to maintain temperature and humidity.

### Fluorescence Microscopy and Imaging

Microscopy was performed on an inverted epifluorescence (Ti2-E, Nikon, Tokyo, Japan) equipped with the Perfect Focus System (PFS) and a Photometrics Prime 95B 25-mm camera. Image acquisition and processing were performed using Nikon Elements AR software. During a time-lapse movie, the specimen was typically imaged at a time interval of 5 min at the focal plane for 2.5-3 h, through channels of phase contrast (200 ms exposure, for cell recognition), blue (DAPI, 200 ms exposure, for phage DNA), and green (GFP, 300 ms exposure, for Gp93-mNeonGreen).

### Next-Generation Sequencing (NGS)

To isolate phage genomic DNA, purified high titer lysates were treated with Benzonase Nuclease (Sigma) for 30 min at 37° C. Phage genomic DNA was extracted with a modified Wizard DNA Clean-Up kit (Promega) protocol. DNA samples were quantified with AccuGreen Broad Range dsDNA quantification kit (Biotium, USA) in a Qubit Fluorometer 2.0.

Purified phage genomic DNA was processed following Illumina DNA Preparation Protocol. Samples were sequenced on a MiSeq system (Illumina) with 300 cycles of paired-end sequencing, and loading concentration of 12 pM. Illumina short reads were downsampled to ˜50-100× coverage and de novo assembled using SPAdes. The sequences of mutant phage strains were aligned to the reference genome in Geneious with the Mauve alignment algorithm to confirm the intended genomic edits.

The isolated orf54 “pseudo knock-out” phage strain (“orf54”) was sequenced using long-read sequencing. DNA samples were processed using SQK-LSK109 kit (Oxford Nanopore Technologies, UK). Libraries were sequenced using an R10.3 flow cell until the desired number of reads was achieved. Oxford Nanopore long reads were filtered for the longest high quality reads using Nanofilt and de novo assembled using Flye.

TABLE 1
Summary of phage mutants engineered by CRISPR-Cas13a.

| | Gene No./ | Identified | | | screening | Isolated |
|---|---|---|---|---|---|---|
| Phage | Genomic site | protein | Modification | Plaque#¹ | % | or not? |
| ϕKZ | Upstream of orf54 | — | Insertion | 17 | 41.2% | Yes |
| | Downstream of | — | Insertion | 16 | 50.0% | |
| | orf120 | | | | | |
| | orf39 | PhuZ | Deletion | 17 | 52.9% | |
| | orf93 | Inner body | Deletion | 20 | 20.0% | |
| | | protein | gp93-mNeonGreen | 22 | 18.2% | |
| | | | fusion | | | |
| | orf241 | Hypothetical | Deletion | 23 | 26.1% | |
| | orf241, orf242 | Hypothetical | Double deletions | 12 | 41.7% | |
| | orf54 | Shell | Deletion | 15 | 20.0% | No |
| | | | mCherry-gp54 fusion | 24 | 8.3% | |
| | | | gp54-mCherry fusion | 11 | 9.1% | |
| | | | gfp11-gp54 fusion | 57 | 7.0% | |
| | orf89, orf90, | Head | Multiple deletions | 17 | 11.8% | |
| | orf91, orf92, orf93 | structural | | | | |
| | | proteins | | | | |
| | orf146 | Structural | Deletion | 86 | 0.0% | |
| | | protein | | | | |
| OMKO1 | Downstream of | — | Deletion | 17 | 11.8% | Yes |
| | the capsid gene | | | | | |
| | Upstream of the | — | Deletion | 89 | 0% | |
| | capsid gene | | | | | |
| PaMx41 | orf24 | Hypothetical | Deletion | 8 | 100.0% | Yes |

¹Number of plaques analyzed by PCR to screen for recombinants.

TABLE 2
Bacterial strains and phages used in this work.

| | | |
|---|---|---|
| Name | Description | Source/Reference |

Bacterial strains

| | | |
|---|---|---|
| PAO1 | P. aeruginosa | Lab collection |
| SDM084 | PAO1 tn7::Lse cas13a | [19] |
| BF# | P. aeruginosa CF clinical | Paul Turner Lab |
| | strains | |

Phages

| | |
|---|---|
| ΦKZ | Alan Davidson Lab |
| JBD30 | Alan Davidson Lab |
| OMKO1 | Paul Turner Lab |
| PaMx41 | Gabriel Guarneros Peña at Centro |
| | de Investigación y de Estudios |
| | Avanzados (KU884563) |

TABLE 3
Sample crRNA sequences used in this work.

| | | | SEQ | |
|---|---|---|---|---|
| | | | ID | |
| Phage | Target | Type | NO: | Sequence (5′-3′) |
| JBD30 | orf38 | VI-A | 3 | acgccaatctctgctacgacggtc |
| ΦKZ | orf54 | VI-A | 4 | tcaaactcaagctactgctcctgc |
| | | | 5 | atgctcgttcagcagatgccctac |
| | | | 6 | ggtatggctggtctactcattgtt |
| | orf120 | | 7 | cttaagccgtgagctggaaagggt |
| | | | 8 | gctgatcttaagccgtgagctgga |
| | | | 9 | agcttgctgatcttaagccgtgag |
| | orf146 | | 10 | ttcatactgggttgaaactaatga |
| | | | 11 | ggcttctgcatttgaagattatgt |
| PaMx41 | orf11 | VI-A | 12 | ccaccctgaatcctcaagaactca |
| | | | 13 | agactggtcgtatctcggctgcta |
| | | | 14 | aaagagcgcgtatatctgaccgcc |
| | | | 15 | gtcaagcgtcagactggtcgtatc |
| | | | 16 | tcaatcttaccgtcaagcgtcaga |
| | | | 17 | ctcggctgctatcatcgctgcgaa |
| | orf04 | I-C | 18 | tgatacgaaggaaattgtcgcaattggcgataga |
| | orf28 | | 19 | atttctgactttaatagggaggccgaattccata |
| | orf14 | I-F | 20 | tctcagcgtaatagtcctgtttcttctggcag |
| | orf42 | | 21 | ttcttctatttctcccagaatctttccgcaat |
| | orf06 | II-A | 22 | tttattactgttgaccctgc |
| | orf20 | | 23 | tttccagcatagttctatac |
| | orf23 | V | 24 | tggagaatgaagaaggtaaggta |
| | orf46 | | 25 | agaatatgccttgcataggatag |

TABLE 4

| | SEQ ID | | | |
|---|---|---|---|---|
| ID | NO: | Description | Name | Sequence (5′-3′) |
| JG003 | 26 | mNeonGreen | mGreen-F | CCAATATTCTCGAGGGTGGCGGTGGCTCGATGGTGAGCAAGGGCG |
| JG004 | 27 | | mGreen-pHERD-R | GACTCTAGAGGATCCCCGGGTACCTTACTTGTACAGCTCGTCC |
| JG010 | 28 | gp93 | IB3-SacI-F | AGCATCGAATTCGAGCTCCATGTCTCTACTTAAAATGCT |
| JG011 | 29 | | IB3-XhoI-R | AGCATCCTCGAGCTTCTCATCATTCTCAGGTG |
| JG075 | 30 | H1 for insertion | pHERD-gp120H1-F | CCATGGGATCTGATAAGAATTCCCTAGGGTAATGCGTCCGACCTATCG |
| JG064 | 31 | at gp120 3′UTR | gp120-muH1stop-R | GATCATTTTTTATTCCTACGTAGGCTCGAGCTAAGGTTTTAAGTCGGCAA |
| | | | | GCTTACC |
| JG029 | 32 | H2 for insertion | gp120-H2-KpnI-F | AGCATCGGTACCCATATGGCTGGAAAGGGTTACCGTTA |
| JG030 | 33 | at gp120 3′UTR | gp120-H2-SalI-R | AGCATCGTCGACCAACAAAACGTGAGACAGGG |
| JG046 | 34 | Check insertion | CheckFP-inphage-R | AGTGCTTCTTCTTCTGGACTAAGTC |
| JG047 | 35 | at gp120 3′ UTR | CheckFP-inphage-F | GTGGTACTATCAGCCTATCCAAAGG |
| JG065 | 36 | AcrVIA1 | AcrVIA1-XhoI-F | AGGCCCCTCGAGCCTACGTAGGAATAAAAAATGATCTACTATATAAAAG |
| JG066 | 37 | | AcrVIA1-NdeI-R | CGGCCCCATATGTTAATTTAGCTCCTCTTTTA |
| JG088 | 38 | Check the | AcrVIA1-mid-F | TCCGTTCGCTCCCGATACGA |
| | | existance of | | |
| | | AcrVIA1 | | |
| JG095 | 39 | H1 for mCherry- | pHERD-Op24NheI-F | GCCTTTTGCTGGCCTTTTGCTCACATAAGTTGAAGACCCGCCACTATATTT |
| | | gp54 fusion | | AGG |
| JG096 | 40 | | Op24H1-AcrVIA-R | CACTTTTAAATCTTTTATATAGTAGATCATTTTAAATACCTTTACGATTAT |
| | | | | GG |
| JG097 | 41 | AcrVIA1 for | Op24H1-AcrVIA-F | CAAGAAACCATAATCGTAAAGGTATTTAAAATGATCTACTATATAAAAG |
| | | mCherry-gp54 | | A |
| JG098 | 42 | fusion | AcrVIA-mCherry-R | CACCATTTTTTATTCCTACGTAGGCCTAGGTTAATTTAGCTCCTCTTTTA |
| JG099 | 43 | H2 for mCherry- | AcrVIA-mCherry-F | AATTAACCTAGGCCTACGTAGGAATAAAAAATGGTGAGCAAGGGCGAGG |
| | | gp54 fusion | | A |
| JG100 | 44 | | pHERD-gp54NheI-R | TGCTCTGCGAGGCTGGCCGATAAGCTAGATGTGGGTCGCAATAGAGAAC |
| | | | | GGAG |
| JG104 | 45 | H1 for gp54- | pHERD-gp54end-F | GCCTTTTGCTGGCCTTTTGCTCACATAAGGAGCAAGTCATCCTAGATGCA |
| JG105 | 46 | gfp11 fusion | gp54end-gfp11-R | CATGTGGTCACGCGAGCCGCCCGACCCGCCGTACCAGGTACCCGGTGCA |
| | | | | T |
| JG106 | 47 | H2 for gp39 | KO39H2-F | ATTAACTTATACTGGAGCCCTTCGG |
| JG107 | 48 | deletion | pHERD-KO39H2-R | TGCTCTGCGAGGCTGGCCGATAAGCTAGCTTTTTAGCAGTGTTCTTTGCC |
| JG050 | 49 | Check gp54 | gp54-mid-R | CAGTGGTCGGAGTCCAATGTAGATC |
| JG108 | 50 | editing | Check-recomb54-F | AGTTTGAGGACCAAGTGTAACACC |
| JG118 | 51 | | Check-KO54-R | GTGGGTTAATTAGGCTACGTACGTG |
| JG110 | 52 | gp54 | pHERD-gp54NheI-F | GCCTTTTGCTGGCCTTTTGCTCACATAAGCTGCATTTTATAAAAATACTG |
| JG111 | 53 | | pHERD-gp54NheI-R | TGCTCTGCGAGGCTGGCCGATAAGCTAGTTAGTACCAGGTACCCGGT |
| JG127 | 54 | | gp54-PstI-F | AGCATCCTGCAGATGGCTGTTAACGAAAACGA |
| JG128 | 55 | | gp54-SacI-R | AGCGTCGAGCTCTTAGTACCAGGTACCCGGTGCATTATAG |
| JG112 | 56 | AcrVIA1 for | AcrVIA-Op24H2-R | TAACATCCCTATCTACCGAGGTATTTCCTATTAATTTAGCTCCTCTTTTA |
| | | gp54 deletion | | |
| | | (pair with | | |
| | | JG097) | | |
| JG113 | 57 | H2 for gp54 | AcrVIA-Op24H2-F | GCAAACAAATTTTAAAAGAGGAGCTAAATTAATAGGAAATACCTCGGTA |
| | | deletion | | GATAGG |
| JG114 | 58 | | pHERD-Op24H2NheI-R | TGCTCTGCGAGGCTGGCCGATAAGCTAGCTTTCTCATCTGAGTTAC |
| JG119 | 59 | H2 for gp93 | KO93H2-F | CAAACATAATGAGGAACCCTTCGG |
| JG120 | 60 | deletion | pHERD-KO93H2-R | TGCTCTGCGAGGCTGGCCGATAAGCTAGCATAAAAGCAGCTGATTGTTTA |
| JG121 | 61 | Check gp93 | Check-KOgp93-R | AGTTAGTACACGCTGTGCCGCT |
| | | deletion | | |
| JG122 | 62 | gp39 | pHERD-gp39-NheI-F | GCCTTTTGCTGGCCTTTTGCTCACATAAGATTATATTAATCACAATGAGG |
| JG123 | 63 | | pHERD-gp39-NheI-R | TGCTCTGCGAGGCTGGCCGATAAGCTAGTTAATCGAATACAAGACCACT |
| | | | | ACT |
| JG124 | 64 | H1 for gp241- | pHERD-KO2412-H1Acr-F | GCCTTTTGCTGGCCTTTTGCTCACATAAGTTAAATAAAGATAACTTCTTTT |
| | | gp242 deletion | | C |
| JG125 | 65 | | KO2412-H1Acr-R | CCTTTTAGTTAATTTAATTTAGCTCCTCTTTTAAAATTTG |
| JG126 | 66 | Check gp241- | Check-KO2412-R | GGCTAAGTCTTTTTCTCGATACTGG |
| | | gp242 deletion | | |
| JG133 | 67 | Operon#24 | pHERD-Op24NheI-F | GCCTTTTGCTGGCCTTTTGCTCACATAAGTACGTGACGGAGCATTCTTAA |
| | | | | ATA |
| JG134 | 68 | | pHERD-Op24NheI-R | TGCTCTGCGAGGCTGGCCGATAAGCTAGCCCGAAGGAGCCGCTATATCA |
| | | | | G |
| JG135 | 69 | H1 for insertion | Omk-capH1-GibsonF | GCCTTTTGCTGGCCTTTTGCTCACATAAGATCGAAGGCGCACTATCCGCT |
| | | at the 3′UTR of | | G |
| JG136 | 70 | the capsid gene | Omk-capmuHl-GibsonR | GATCATTTTTTATTCCTACGTAGGGCTAGCCTAAGGTTTTAAGTCGGCAA |
| | | in OMKO1 | | GCTTACC |
| JG137 | 71 | H1 for insertion | pHERD-Op24InH1-F | GCCTTTTGCTGGCCTTTTGCTCACATAAGGGAGCATTCTTAAATAAATTA |
| JG138 | 72 | at gp54 5′UTR | Op24InH1-RBS-R | GATCATTGTATATTCCTATTTGTAATCTCGAGCGTAACGAACACTATGTT |
| JG139 | 73 | AcrVIA1 for | gp53RBS-AcrVIA1-F | CTCGAGATTACAAATAGGAATATACAATGATCTACTATATAAAAGA |
| | | insertion at gp54 | | |
| JG140 | 74 | 5′UTR | AcrVIA1-Op24InH2-R | CGCTATAATCTGCTATCAGCAGATCCTAGGTTAATTTAGCTCCTCTTTTA |
| JG141 | 75 | H2 for insertion | AcrVIA1-Op24InH2-F | AGGAGCTAAATTAACCTAGGATCTGCTGATAGCAGATTATAGCG |
| JG142 | 76 | at gp54 5′UTR | pHERD-Op24InH2-R | TGCTCTGCGAGGCTGGCCGATAAGCTAGGTTGTAACGGTTCCGTAG |
| JG146 | 77 | H1 for gp146 | pHERD-KOgp146H1-F | GCCTTTTGCTGGCCTTTTGCTCACATAAGAGCTATTGCAAATGCGGTACC |
| JG147 | 78 | deletion | KOgp146H1-AcrR | TAGATCATTTTTATTTCCTTGCTGTATAAGAACATTATACTACTTCCTCAA |
| | | | | GTTCG |
| JG148 | 79 | Check gp146 | Check-gp146edit-R | AATCTATCCCAGTTCAAGCTAAGCC |
| | | deletion | | |
| JG152 | 80 | AcrVIA1 for | gp145RBS-AcrVIA1-F | TTCTTATACAGCAAGGAAATAAAAATGATCTACTATATAAAAGAT |
| JG153 | 81 | gp146 deletion | AcrVIA1-Op63InH2-R | GGAGGGCAATATGTCGTTTTAATTTAGCTCCTCTTTTAAAAT |
| JG154 | 82 | H2 for gp146 | AcrVIA1-Op63InH2-F | AAAGAGGAGCTAAATTAAAACGACATATTGCCCTCCCTTCGG |
| JG155 | 83 | deletion | pHERD-Op63InH2-R | TGCTCTGCGAGGCTGGCCGATAAGCTAGATTCCATTTATTATTCAATTAA |
| | | | | TAGCT |
| JG156 | 84 | gp146 | pMMB-gp146-GbF | GGAAACAGAATTAATTAAGCTTGCATGCCTGCAGATGGCTTCTGCATTTG |
| | | | | AAGA |
| JG157 | 85 | | pMMB-gp146-GbR | CCGCCAAAACAGCCAAGCTGAATTCGAGCTCTTAAATAACAGCACCTTT |
| | | | | GG |
| JG160 | 86 | H2 for gp241 | AcrVIA1-KO241H2-F | CAAACAAATTTTAAAAGAGGAGCTAAATTAATAAGAGCATATGGCCCTC |
| | | deletion | | CCTTCGG |
| JG161 | 87 | | pHERD-KO241H2-R | TGCTCTGCGAGGCTGGCCGATAAGCTAGTAATAGTGGTATTAACTTGTAG |
| JG162 | 88 | Check gp241 | Check-KOgp241-R | CGGGTGTAATCGTACGTAGATCAATCGC |
| | | deletion | | |
| JG166 | 89 | H2 for gp241- | KO2412-H2-F | GAGGAGCTAAATTAAATTAACTAAAAGGTAAAAAAATGAATAATC |
| JG167 | 90 | gp242 deletion | pHERD-KO2412-H2-R | TGCTCTGCGAGGCTGGCCGATAAGCTAGCATCCTTATTATGGAAGCTATA |
| JG168 | 91 | Check orf24 | Check-PaM24-R | CTCGTGGTGATGTTAACCCTAGGG |
| JG169 | 92 | deletion in | Check-PaM24-F | CAACTCTGAATTCCGAACTGAGGCC |
| | | PaMx41 | | |
| JG170 | 93 | Check gp93 | Check-KOgp93-F | ACTCTTGTATATCCAATCTGTAGCGG |
| | | deletion | | |
| JG173 | 94 | gp39 | gp39-SacI-F | ACCCATGGGATCTGATAAGAATTCGAGCTCATGATGTCTAAAGTAAAAA |
| | | | | CTCG |
| JG174 | 95 | | gp39-KpnI-R | CAGGTCGACTCTAGAGGATCCCCGGGTACCTTAATCGAATACAAGACCA |
| | | | | CTACT |
| JG175 | 96 | H1 for | pHERD-KOop42H1-F | GCCTTTTGCTGGCCTTTTGCTCACATAAGTAACCTACCTTCTTTTACTAAT |
| | | Operon#42 | | GCAGAATAG |
| JG176 | 97 | deletion | KOop42H1-R | CTTTTAAATCTTTTATATAGTAGATCATTTTTCTCTTCCTATATGGGTTTGC |
| | | | | AGAG |
| JG178 | 98 | gfp1-10 | pHERD-gfp10-GbF | ACCCATGGGATCTGATAAGAATTCGAGCTCATGGTGAGCAAGGGCGAGG |
| | | | | A |
| JG179 | 99 | | pHERD-gfp10-GbR | GGCCAGTGCCAAGCTTGCATGCCTGCAGCTTCTCGTTGGGGTCTTTGCTC |
| JG180 | 100 | Check gfp11- | gfp11-mid-F | TGGTCCTTCATGAGTATGTAAATGC |
| | | gp54 and gp54- | | |
| | | gfp11 fusion | | |
| JG181 | 101 | Check gp39 | Check-KOgp39-R | CTTCAGCCCATTTGTCTACCCG |
| | | deletion | | |
| JG182 | 102 | H1 for gp93- | gp93-mNGH1-Gb-F | GCCTTTTGCTGGCCTTTTGCTCACATAAGAATCCGAAGACTATTGCAGAC |
| | | mNeonGreen | | TTCA |
| JG183 | 103 | fusion | gp93-mNGH1-Gb-R | CTATATGGGTTTGCGGTTCCTCATTATGTTTGTTATTACTTGTACAGCTCG |
| | | | | TCCAT |
| JG186 | 104 | Check | Check-KOop42-F | CACCAATAAAGTCACCAGTACGTCC |
| | | Operon#42 | | |
| | | deletion | | |
| JG187 | 105 | Check gp93- | gp93-mid-F | GCCCTAGCTGATTGGAATCCTGACG |
| | | mNeonGreen | | |
| | | fusion | | |
| oSDM455 | 106 | pHERD30T- | Lse_crRNA_V3_Top | ACTCTCTACTGTTTCTCCATGGTAAGAGACTACCTCTATATGAAAGAGGA |
| | | crRNA Version | | CTAAAACCGA |
| oSDM456 | 107 | 3 | Lse_crRNA_V3_Bot | CGGGTACCGAGCTCGAATTCGAGACCGTACGTACGTGGTCTCGGTTTTAG |
| | | | | TCCTCTTTCA |
| oSDM457 | 108 | pHERD30T- | p30T_TSS-R | ATGGAGAAACAGTAGAGAGTTG |
| oSDM458 | 109 | crRNA Version | p30T_TSS-F | GAATTCGAGCTCGGTACC |
| oSDM465 | 110 | 2 | LseCas13 crRNA V2 F | ACTCTCTACTGTTTCTCCATGGTAAGAGACTACCTCTATATGAAAGAGGA |
| | | | | CTAAAACCGAGACCACGTACGTACGGTC |
| oSDM466 | 111 | | LseCas13 crRNA V2 R | CGGGTACCGAGCTCGAATTCGTTTTTGTGGAGATTCATATTCTCCAAGTC |
| | | | | TCTTACCGGAGACCGTACGTACGTGGTC |

TABLE 5
Host range assay of engineered OMKO1 variants.

| | | Phage | | | | | |
|---|---|---|---|---|---|---|---|
| | | OMKO1 | | OMKO1::Acr | | OMKO1::Acr-BC | |
| Host | PA01 | 85 | 93 | 87 | 93 | 85 | 94 |
| | FB-4 | 25 | 35 | 25 | 37 | 25 | 33 |
| | FB-5 | 1 | 5 | 2 | 4 | 2 | 6 |
| | FB-15 | 0 | 2 | 0 | 2 | 0 | 5 |
| | FB-19 | 62 | 73 | 69 | 77 | 69 | 77 |
| | FB-52 | 89 | 93 | 91 | 94 | 90 | 94 |
| | FB-62 | 88 | 95 | 89 | 94 | 86 | 93 |
| | FB-63 | 0 | 59 | 0 | 59 | 0 | 60 |
| | FB-66 | 68 | 87 | 70 | 87 | 48* | 86 |
| | FB-68 | 4 | 23 | 4 | 21 | 3 | 21 |
| | FB-71 | 9 | 59 | 10 | 60 | 4* | 46* |
| | FB-72 | 3 | 13 | 6 | 14 | 4 | 14 |
| | FB-78 | 81 | 91 | 83 | 91 | 80 | 91 |
| | FB-81 | 79 | 92 | 81 | 93 | 79 | 92 |
| | FB-83 | 79 | 86 | 73 | 87 | 67 | 86 |
| | FB-87 | 4 | 42 | 5 | 43 | 1 | 30* |
| | FB-89 | 1 | 34 | 1 | 52* | 1 | 62* |
| | FB-92 | 0 | 37 | 2 | 55* | 2 | 64* |
| | FB-94 | 8 | 93 | 6 | 93 | 8 | 93 |
| | FB-96 | 84 | 94 | 86 | 94 | 85 | 93 |
| | FB-98 | 80 | 92 | 83 | 92 | 76 | 91 |
| | FB-99 | 85 | 93 | 85 | 93 | 84 | 92 |
| | | 0.01 | 1 | 0.01 | 1 | 0.01 | 1 |

MOI

Host ranges were determined by microplate liquid assay at MOI of 0.01 and 1 on 22 P. aeruginosa clinical strains. The values are presented as the mean liquid assay scores across three independent experiments.
Asterisks (*) indicate significant difference between WT and engineered strains as determined by Students' T-test (p < 0.05). The color intensity of each phage-host combination reflects the liquid assay score, which represents how well the phage strain can repress the growth of a given bacterial host. No inhibition of bacterial growth is reflected by a liquid assay score of 0, and complete suppression would result in a score of 100.

### REFERENCES

1. Kortright, K. E., Chan, B. K., Koff, J. L., and Turner, P. E. (2019). Phage Therapy: A Renewed Approach to Combat Antibiotic-Resistant Bacteria. Cell Host Microbe 25, 219-232.
2. Pires, D. P., Cleto, S., Sillankorva, S., Azeredo, J., and Lu, T. K. (2016). Genetically Engineered Phages: a Review of Advances over the Last Decade. Microbiol Mol Biol Rev 80, 523-543.
3. Doss, J., Culbertson, K., Hahn, D., Camacho, J., and Barekzi, N. (2017). A Review of Phage Therapy against Bacterial Pathogens of Aquatic and Terrestrial Organisms. Viruses 9.
4. Nobrega, F. L., Costa, A. R., Kluskens, L. D., and Azeredo, J. (2015). Revisiting phage therapy: new applications for old resources. Trends Microbiol 23, 185-191.
5. Lusiak-Szelachowska, M., Zaczek, M., Weber-Dabrowska, B., Miedzybrodzki, R., Klak, M., Fortuna, W., Letkiewicz, S., Rogoz, P., Szufnarowski, K., Jonczyk-Matysiak, E., et al. (2014). Phage Neutralization by Sera of Patients Receiving Phage Therapy. Viral Immunol 27, 295-304.
6. Weber-Dabrowska, B., Jonczyk-Matysiak, E., Zaczek, M., Lobocka, M., Lusiak-Szelachowska, M., and Gorski, A. (2016). Bacteriophage Procurement for Therapeutic Purposes. Front Microbiol 7, 1177.
7. Lu, T. K., and Koeris, M. S. (2011). The next generation of bacteriophage therapy. Curr Opin Microbiol 14, 524-531.
8. Lenneman, B. R., Fembach, J., Loessner, M. J., Lu, T. K., and Kilcher, S. (2020). Enhancing phage therapy through synthetic biology and genome engineering. Curr Opin Biotechnol 68, 151-159.
9. Ando, H., Lemire, S., Pires, D. P., and Lu, T. K. (2015). Engineering Modular Viral Scaffolds for Targeted Bacterial Population Editing. Cell Syst 1, 187-196.
10. Mahichi, F., Synnott, A. J., Yamamichi, K., Osada, T., and Tanji, Y. (2009). Site-specific recombination of T2 phage using IP008 long tail fiber genes provides a targeted method for expanding host range while retaining lytic activity. FEMS Microbiol Lett 295, 211-217.
11. Matsuda, T., Freeman, T. A., Hilbert, D. W., Duff, M., Fuortes, M., Stapleton, P. P., and Daly, J. M. (2005). Lysis-deficient bacteriophage therapy decreases endotoxin and inflammatory mediator release and improves survival in a murine peritonitis model. Surgery 137, 639-646.
12. Monteiro, R., Pires, D. P., Costa, A. R., and Azeredo, J. (2019). Phage Therapy: Going Temperate?Trends Microbiol 27, 368-378.
13. Kilcher, S., and Loessner, M. J. (2019). Engineering Bacteriophages as Versatile Biologics. Trends Microbiol 27, 355-367.
14. Marinelli, L. J., Hatfull, G. F., and Piuri, M. (2012). Recombineering: A powerful tool for modification of bacteriophage genomes. Bacteriophage 2, 5-14.
15. Deveau, H., Gameau, J. E., and Moineau, S. (2010). CRISPR/Cas system and its role in phage-bacteria interactions. Annu Rev Microbiol 64, 475-493.
16. Hille, F., Richter, H., Wong, S. P., Bratovic, M., Ressel, S., and Charpentier, E. (2018). The Biology of CRISPR-Cas: Backward and Forward. Cell 172, 1239-1259.
17. Samson, J. E., Magadan, A. H., Sabri, M., and Moineau, S. (2013). Revenge of the phages: defeating bacterial defences. Nat Rev Microbiol 11, 675-687.
18. Malone, L. M., Birkholz, N., and Fineran, P. C. (2020). Conquering CRISPR: how phages overcome bacterial adaptive immunity. Curr Opin Biotechnol 68, 30-36.
19. Mendoza, S. D., Nieweglowska, E. S., Govindarajan, S., Leon, L. M., Berry, J. D., Tiwari, A., Chaikeeratisak, V., Pogliano, J., Agard, D. A., and Bondy-Denomy, J. (2020). A bacteriophage nucleus-like compartment shields DNA from CRISPR nucleases. Nature 577, 244-248.
20. Malone, L. M., Warring, S. L., Jackson, S. A., Warnecke, C., Gardner, P. P., Gumy, L. F., and Fineran, P. C. (2020). A jumbo phage that forms a nucleus-like structure evades CRISPR-Cas DNA targeting but is vulnerable to type III RNA-based immunity. Nat Microbiol 5, 48-55.
21. Guan, J., and Bondy-Denomy, J. (2020). Intracellular Organization by Jumbo Bacteriophages. J Bacteriol 203.
22. Abudayyeh, O. O., Gootenberg, J. S., Konermann, S., Joung, J., Slaymaker, I. M., Cox, D. B., Shmakov, S., Makarova, K. S., Semenova, E., Minakhin, L., et al. (2016). C2c2 is a single-component programmable RNA-guided RNA-targeting CRISPR effector. Science 353, aaf5573.
23. Meeske, A. J., Jia, N., Cassel, A. K., Kozlova, A., Liao, J., Wiedmann, M., Patel, D. J., and Marraffini, L. A. (2020). A phage-encoded anti-CRISPR enables complete evasion of type VI-A CRISPR-Cas immunity. Science 369, 54-59.
24. Abudayyeh, O. O., Gootenberg, J. S., Essletzbichler, P., Han, S., Joung, J., Belanto, J. J., Verdine, V., Cox, D. B. T., Kellner, M. J., Regev, A., et al. (2017). RNA targeting with CRISPR-Cas13. Nature 550, 280-284.
25. Al-Shayeb, B., Sachdeva, R., Chen, L. X., Ward, F., Munk, P., Devoto, A., Castelle, C. J., Olm, M. R., Bouma-Gregson, K., Amano, Y., et al. (2020). Clades of huge phages from across Earth's ecosystems. Nature 578, 425-431.
26. Aylett, C. H., Izore, T., Amos, L. A., and Lowe, J. (2013). Structure of the tubulin/FtsZ-like protein TubZ from Pseudomonas bacteriophage PhiKZ. J Mol Biol 425, 2164-2173.
27. Chaikeeratisak, V., Nguyen, K., Egan, M. E., Erb, M. L., Vavilina, A., and Pogliano, J. (2017). The Phage Nucleus and Tubulin Spindle Are Conserved among Large Pseudomonas Phages. Cell Rep 20, 1563-1571.
28. Chaikeeratisak, V., Khanna, K., Nguyen, K. T., Sugie, J., Egan, M. E., Erb, M. L., Vavilina, A., Nonejuie, P., Nieweglowska, E., Pogliano, K., et al. (2019). Viral Capsid Trafficking along Treadmilling Tubulin Filaments in Bacteria. Cell 177, 1771-1780 e1712.
29. Kraemer, J. A., Erb, M. L., Waddling, C. A., Montabana, E. A., Zehr, E. A., Wang, H., Nguyen, K., Pham, D. S., Agard, D. A., and Pogliano, J. (2012). A phage tubulin assembles dynamic filaments by an atypical mechanism to center viral DNA within the host cell. Cell 149, 1488-1499.
30. Chaikeeratisak, V., Nguyen, K., Khanna, K., Brilot, A. F., Erb, M. L., Coker, J. K., Vavilina, A., Newton, G. L., Buschauer, R., Pogliano, K., et al. (2017). Assembly of a nucleus-like structure during viral replication in bacteria. Science 355, 194-197.
31. Wu, W., Thomas, J. A., Cheng, N., Black, L. W., and Steven, A. C. (2012). Bubblegrams reveal the inner body of bacteriophage phiKZ. Science 335, 182.
32. Thomas, J. A., Weintraub, S. T., Wu, W., Winkler, D. C., Cheng, N., Steven, A. C., and Black, L. W. (2012). Extensive proteolysis of head and inner body proteins by a morphogenetic protease in the giant Pseudomonas aeruginosa phage phiKZ. Mol Microbiol 84, 324-339.
33. Chan, B. K., Turner, P. E., Kim, S., Mojibian, H. R., Elefteriades, J. A., and Narayan, D. (2018). Phage treatment of an aortic graft infected with Pseudomonas aeruginosa. Evol Med Public Health 2018, 60-66.
34. Sepulveda-Robles, O., Kameyama, L., and Guarneros, G. (2012). High diversity and novel species of Pseudomonas aeruginosa bacteriophages. Appl Environ Microbiol 78, 4510-4515.
35. Cruz-Plancarte, I., Cazares, A., and Guarneros, G. (2016). Genomic and Transcriptional Mapping of PaMx41, Archetype of a New Lineage of Bacteriophages Infecting Pseudomonas aeruginosa. Appl Environ Microbiol 82, 6541-6547.
36. Skennerton, C. T., Angly, F. E., Breitbart, M., Bragg, L., He, S., McMahon, K. D., Hugenholtz, P., and Tyson, G. W. (2011). Phage encoded H-NS: a potential achilles heel in the bacterial defence system. PLoS One 6, e20095.
37. Pul, U., Wurm, R., Arslan, Z., Geissen, R., Hofmann, N., and Wagner, R. (2010). Identification and characterization of E. coli CRISPR-cas promoters and their silencing by H-NS. Mol Microbiol 75, 1495-1512.
38. Hampton, H. G., Watson, B. N. J., and Fineran, P. C. (2020). The arms race between bacteria and their phage foes. Nature 577, 327-336.
39. Vlot, M., Houkes, J., Lochs, S. J. A., Swarts, D. C., Zheng, P., Kunne, T., Mohanraju, P., Anders, C., Jinek, M., van der Oost, J., et al. (2018). Bacteriophage DNA glucosylation impairs target DNA binding by type I and II but not by type V CRISPR-Cas effector complexes. Nucleic Acids Res 46, 873-885.
40. Bryson, A. L., Hwang, Y., Sherrill-Mix, S., Wu, G. D., Lewis, J. D., Black, L., Clark, T. A., and Bushman, F. D. (2015). Covalent Modification of Bacteriophage T4 DNA Inhibits CRISPR-Cas9. mBio 6, e00648.
41. Liu, Y., Dai, L., Dong, J., Chen, C., Zhu, J., Rao, V. B., and Tao, P. (2020). Covalent Modifications of the Bacteriophage Genome Confer a Degree of Resistance to Bacterial CRISPR Systems. J Virol 94.
42. Davidson, A. R., Lu, W. T., Stanley, S. Y., Wang, J., Mejdani, M., Trost, C. N., Hicks, B. T., Lee, J., and Sontheimer, E. J. (2020). Anti-CRISPRs. Protein Inhibitors of CRISPR-Cas Systems. Annu Rev Biochem 89, 309-332.
43. van Beljouw, S. P. B., Haagsma, A. C., Rodriguez-Molina, A., van den Berg, D. F., Vink, J. N. A., and Brouns, S. J. J. (2021). The gRAMP CRISPR-Cas effector is an RNA endonuclease complexed with a caspase-like peptidase. Science 373, 1349-1353.
44. Ozcan, A., Krajeski, R., Ioannidi, E., Lee, B., Gardner, A., Makarova, K. S., Koonin, E. V., Abudayyeh, O. O., and Gootenberg, J. S. (2021). Programmable RNA targeting with the single-protein CRISPR effector Cas7-11. Nature 597, 720-725.
45. Mayo-Munoz, D., He, F., Jorgensen, J. B., Madsen, P. K., Bhoobalan-Chitty, Y., and Peng, X. (2018). Anti-CRISPR-Based and CRISPR-Based Genome Editing of Sulfolobus islandicus Rod-Shaped Virus 2. Viruses 10.
46. Krylov, V. N., Smirnova, T. A., Minenkova, I. B., Plotnikova, T. G., Zhazikov, I. Z., and Khrenova, E. A. (1984). Pseudomonas bacteriophage phi KZ contains an inner body in its capsid. Can J Microbiol 30, 758-762.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of introducing a genetic modification at a locus within the genome of a bacteriophage, the method comprising:
i) providing a first population of bacterial cells comprising a polynucleotide and the bacteriophage, wherein the polynucleotide comprises a coding sequence for an anti-CRISPR protein that inhibits an RNA-targeting CRISPR-Cas system in the bacterial cells, flanked by two homology arms comprising nucleotide sequence homology to the bacteriophage locus;
ii) culturing the bacterial cells under conditions conducive to homologous recombination between the polynucleotide and the bacteriophage genome, and obtaining a lysate from the cultured cells;
iii) infecting a second population of bacterial cells comprising the RNA-targeting CRISPR-Cas system with the lysate; and
iv) isolating modified bacteriophage from the contacted population of CRISPR bacterial cells.

2. The method of claim 1, wherein the bacteriophage is a lytic bacteriophage and the isolating comprises isolating recombinant plaques from the contacted population of CRISPR bacterial cells.

3. The method of claim 1, further comprising verifying the integration of the coding sequence for the CRISPR inhibitor at the locus within the bacteriophage genome.

4. The method of claim 1 or 3, wherein the RNA-targeting CRISPR-Cas system comprises Cas13a.

5. The method of claim 1 or 4, wherein one or both of the homology arms are at least about 50, 100, 150, 200, 250, 300, 350, 400, 450, 500, 550, 600, or more nucleotides in length.

6. The method of any one of claims 1 to 5, wherein the bacteriophage is selected from the group consisting of ΦKZ, OMKO1, and PaMx41.

7. The method of any one of claims 1 to 6, wherein the integration of the CRISPR inhibitor into the bacteriophage genome at the locus introduces a genetic modification at the locus.

8. The method of claim 7, wherein the genetic modification is a deletion.

9. The method of claim 7, wherein the genetic modification is an insertion.

10. The method of claim 7, wherein the genetic modification is a nucleotide substitution.

11. The method of any one of claims 1 to 10, wherein the RNA-targeting CRISPR-Cas system comprises a crRNA comprising one or more mismatches between two direct repeat sequences and/or a truncation of 1, 2, 3, 4, 5, 6, 7, 8, 9, 10 or more nucleotides within a direct repeat at the 5′ and/or 3′ end of the crRNA.

12. The method of any one of claims 1 to 10, wherein the RNA-targeting CRISPR-Cas system comprises a crRNA containing one direct repeat sequence.

13. The method of any one of claims 1 to 12, wherein the anti-CRISPR protein is AcrVIA1.

14. The method of any one of claims 1 to 13, wherein the crRNA targets orf120 or orf146, or an ortholog thereof.

15. The method of any one of claims 1 to 14, wherein the locus is from phiKZ phage and is selected from the group consisting of orf39, orf54, orf120, orf146, orf93, orf241, orf242, orf89, orf90, orf91, orf92, orf93, and orthologs thereof.

16. The method of any one of claims 1 to 15, wherein first population of bacterial cells comprises the bacteriophage prior to comprising the polynucleotide.

17. The method of any one of claims 1 to 15, wherein first population of bacterial cells comprises the bacteriophage subsequent to comprising the polynucleotide.

18. The method of any one of claims 1 to 17, wherein the polynucleotide and flanking homology arms are present in an editing plasmid, and wherein the editing plasmid is introduced into the first population of bacterial cells by electroporation.

19. The method of any one of claims 1 to 18, wherein the second population of bacterial cells comprises the RNA-targeting CRISPR-Cas system prior to being contacted with the lysate.

20. The method of any one of claims 1 to 18, wherein the second population of bacterial cells comprises the RNA-targeting CRISPR-Cas system subsequent to being contacted with the lysate.

21. A modified bacteriophage produced using the method of any one of claims 1 to 15.

22. A bacterial cell comprising the modified bacteriophage of claim 21.

23. A modified bacteriophage comprising a coding sequence for an anti-CRISPR protein integrated into the bacteriophage genome at a locus that is not essential for phage replication, wherein the anti-CRISPR protein can inhibit an RNA-targeting CRISPR-Cas system, and wherein the locus also comprises a deletion, insertion, or mutation relative to an otherwise equivalent, non-modified bacteriophage.

24. The modified bacteriophage of claim 23, wherein the anti-CRISPR protein is AcrVIA1.

25. The modified bacteriophage of claim 23 or 24, wherein the bacteriophage is selected from the group consisting of ΦKZ, OMKO1, and PaMx41.

26. The modified bacteriophage of any one of claims 23 to 25, wherein the locus is selected from the group consisting of orf39, orf54, orf120, orf146, orf93, orf241, orf242, orf89, orf90, orf91, orf92, orf93, and orthologs thereof.

27. A bacterial cell comprising the modified bacteriophage of any one of claims 23 to 26.

28. A bacterial cell comprising a polynucleotide and a bacteriophage, wherein the polynucleotide comprises a coding sequence for an anti-CRISPR protein that inhibits an RNA-targeting CRISPR-Cas system, flanked by two homology arms comprising substantial nucleotide sequence identity to a locus within the genome of the bacteriophage.

29. The bacterial cell of claim 28, wherein the anti-CRISPR protein is AcrVIA1.

30. The bacterial cell of claim 28 or 29, wherein the RNA-targeting CRISPR-Cas system comprises Cas13a.


SEQUENCE LISTING (115 sequences)

SEQ ID NO: 1 - protein, 232 aa, Listeriophage Phi-LS46
  REGION 1..232: note = Description of sequence: AcrVIA1 coding sequence
MIYYIKDLKVKGKIFENLMNKEAVEGLITFLKKAEFEIYSRENYSKYNKWFEMWKSPTSSLVFWKNYSFRCHLLFVIEKDGECLGIPASVFESVLQIYLADPFAPDTKELFVEVCNLYECLADVTVVEHFEAEESAWHKLTHNETEVSKRVYSKDDDELLKYIPEFLDTIATNKKSQKYNQIQGKIQEINKEIATLYESSEDYIFTEYVSNLYRESAKLEQHSKQILKEELN

SEQ ID NO: 2 - DNA, 699 bp, Listeriophage Phi-LS46
  misc_feature 1..699: note = Description of sequence: AcrVIA1
  source 1..699: mol_type = genomic DNA
atgatctactatataaaagatttaaaagtgaaaggaaaaatatttgaaaatctaatgaacaaagaggctgtagaaggattaattacttttttaaagaaagcggaatttgagatatactcaagagaaaattattcaaaatacaacaaatggtttgaaatgtggaaaagcccaacttcgagccttgtgttttggaaaaattatagttttcgctgtcatcttctttttgtcatagaaaaagatggtgaatgccttggaattcctgcatctgtttttgaatctgtacttcaaatttaccttgcggatccgttcgctcccgatacgaaagaactttttgttgaggtttgtaatttatatgaatgtttagcggatgtcactgtcgtagaacattttgaagcggaagaatcagcgtggcataaattaacccataatgagaccgaagtatcaaaaagagtctatagtaaagatgatgacgaacttcttaaatatattccagaatttcttgacaccatagcgacaaacaagaaaagtcaaaaatacaatcaaattcaaggaaaaatacaagaaattaataaggaaatagctacactttatgaatcgtcagaggattatatatttactgaatatgttagtaatttatatagagagtctgcaaagttggagcaacacagcaaacaaattttaaaagaggagctaaattaa

SEQ ID NO: 3 - RNA, 24 nt, synthetic construct
  misc_feature 1..24: note = Description of sequence: crRNA sequence
  source 1..24: mol_type = other RNA
acgccaatctctgctacgacggtc

SEQ ID NO: 4 - RNA, 24 nt, synthetic construct
  misc_feature 1..24: note = Description of sequence: crRNA sequence
  source 1..24: mol_type = other RNA
tcaaactcaagctactgctcctgc

SEQ ID NO: 5 - RNA, 24 nt, synthetic construct
  misc_feature 1..24: note = Description of sequence: crRNA sequence
  source 1..24: mol_type = other RNA
atgctcgttcagcagatgccctac

SEQ ID NO: 6 - RNA, 24 nt, synthetic construct
  misc_feature 1..24: note = Description of sequence: crRNA sequence
  source 1..24: mol_type = other RNA
ggtatggctggtctactcattgtt

SEQ ID NO: 7 - RNA, 24 nt, synthetic construct
  misc_feature 1..24: note = Description of sequence: crRNA sequence
  source 1..24: mol_type = other RNA
cttaagccgtgagctggaaagggt

SEQ ID NO: 8 - RNA, 24 nt, synthetic construct
  misc_feature 1..24: note = Description of sequence: crRNA sequence
  source 1..24: mol_type = other RNA
gctgatcttaagccgtgagctgga

SEQ ID NO: 9 - RNA, 24 nt, synthetic construct
  misc_feature 1..24: note = Description of sequence: crRNA sequence
  source 1..24: mol_type = other RNA
agcttgctgatcttaagccgtgag

SEQ ID NO: 10 - RNA, 24 nt, synthetic construct
  misc_feature 1..24: note = Description of sequence: crRNA sequence
  source 1..24: mol_type = other RNA
ttcatactgggttgaaactaatga

SEQ ID NO: 11 - RNA, 24 nt, synthetic construct
  misc_feature 1..24: note = Description of sequence: crRNA sequence
  source 1..24: mol_type = other RNA
ggcttctgcatttgaagattatgt

SEQ ID NO: 12 - RNA, 24 nt, synthetic construct
  misc_feature 1..24: note = Description of sequence: crRNA sequence
  source 1..24: mol_type = other RNA
ccaccctgaatcctcaagaactca

SEQ ID NO: 13 - RNA, 24 nt, synthetic construct
  misc_feature 1..24: note = Description of sequence: crRNA sequence
  source 1..24: mol_type = other RNA
agactggtcgtatctcggctgcta

SEQ ID NO: 14 - RNA, 24 nt, synthetic construct
  misc_feature 1..24: note = Description of sequence: crRNA sequence
  source 1..24: mol_type = other RNA
aaagagcgcgtatatctgaccgcc

SEQ ID NO: 15 - RNA, 24 nt, synthetic construct
  misc_feature 1..24: note = Description of sequence: crRNA sequence
  source 1..24: mol_type = other RNA
gtcaagcgtcagactggtcgtatc

SEQ ID NO: 16 - RNA, 24 nt, synthetic construct
  misc_feature 1..24: note = Description of sequence: crRNA sequence
  source 1..24: mol_type = other RNA
tcaatcttaccgtcaagcgtcaga

SEQ ID NO: 17 - RNA, 24 nt, synthetic construct
  misc_feature 1..24: note = Description of sequence: crRNA sequence
  source 1..24: mol_type = other RNA
ctcggctgctatcatcgctgcgaa

SEQ ID NO: 18 - RNA, 34 nt, synthetic construct
  misc_feature 1..34: note = Description of sequence: crRNA sequence
  source 1..34: mol_type = other RNA
tgatacgaaggaaattgtcgcaattggcgataga

SEQ ID NO: 19 - RNA, 34 nt, synthetic construct
  misc_feature 1..34: note = Description of sequence: crRNA sequence
  source 1..34: mol_type = other RNA
atttctgactttaatagggaggccgaattccata

SEQ ID NO: 20 - RNA, 32 nt, synthetic construct
  misc_feature 1..32: note = Description of sequence: crRNA sequence
  source 1..32: mol_type = other RNA
tctcagcgtaatagtcctgtttcttctggcag

SEQ ID NO: 21 - RNA, 32 nt, synthetic construct
  misc_feature 1..32: note = Description of sequence: crRNA sequence
  source 1..32: mol_type = other RNA
ttcttctatttctcccagaatctttccgcaat

SEQ ID NO: 22 - RNA, 20 nt, synthetic construct
  misc_feature 1..20: note = Description of sequence: crRNA sequence
  source 1..20: mol_type = other RNA
tttattactgttgaccctgc

SEQ ID NO: 23 - RNA, 20 nt, synthetic construct
  misc_feature 1..20: note = Description of sequence: crRNA sequence
  source 1..20: mol_type = other RNA
tttccagcatagttctatac

SEQ ID NO: 24 - RNA, 23 nt, synthetic construct
  misc_feature 1..23: note = Description of sequence: crRNA sequence
  source 1..23: mol_type = other RNA
tggagaatgaagaaggtaaggta

SEQ ID NO: 25 - RNA, 23 nt, synthetic construct
  misc_feature 1..23: note = Description of sequence: crRNA sequence
  source 1..23: mol_type = other RNA
agaatatgccttgcataggatag

SEQ ID NO: 26 - DNA, 45 bp, synthetic construct
  misc_feature 1..45: note = Description of sequence: Description: Name: mGreen-F; ID: JG003
  source 1..45: mol_type = other DNA
ccaatattctcgagggtggcggtggctcgatggtgagcaagggcg

SEQ ID NO: 27 - DNA, 43 bp, synthetic construct
  misc_feature 1..43: note = Description of sequence: Description: Name: mGreen-pHERD-R; ID: JG004
  source 1..43: mol_type = other DNA
gactctagaggatccccgggtaccttacttgtacagctcgtcc

SEQ ID NO: 28 - DNA, 39 bp, synthetic construct
  misc_feature 1..39: note = Description of sequence: Description: Name: IB3-SacI-F; ID: JG010
  source 1..39: mol_type = other DNA
agcatcgaattcgagctccatgtctctacttaaaatgct

SEQ ID NO: 29 - DNA, 32 bp, synthetic construct
  misc_feature 1..32: note = Description of sequence: Description: Name: IB3-XhoI-R; ID: JG011
  source 1..32: mol_type = other DNA
agcatcctcgagcttctcatcattctcaggtg

SEQ ID NO: 30 - DNA, 48 bp, synthetic construct
  misc_feature 1..48: note = Description of sequence: Description: Name: pHERD-gp120H1-F; ID: JG075
  source 1..48: mol_type = other DNA
ccatgggatctgataagaattccctagggtaatgcgtccgacctatcg

SEQ ID NO: 31 - DNA, 57 bp, synthetic construct
  misc_feature 1..57: note = Description of sequence: Description: Name: gp120-muH1stop-R; ID: JG064
  source 1..57: mol_type = other DNA
gatcattttttattcctacgtaggctcgagctaaggttttaagtcggcaagcttacc

SEQ ID NO: 32 - DNA, 38 bp, synthetic construct
  misc_feature 1..38: note = Description of sequence: Description: Name: gp120-H2-KpnI-F; ID: JG029
  source 1..38: mol_type = other DNA
agcatcggtacccatatggctggaaagggttaccgtta

SEQ ID NO: 33 - DNA, 32 bp, synthetic construct
  misc_feature 1..32: note = Description of sequence: Description: Name: gp120-H2-SalI-R; ID: JG030
  source 1..32: mol_type = other DNA
agcatcgtcgaccaacaaaacgtgagacaggg

SEQ ID NO: 34 - DNA, 25 bp, synthetic construct
  misc_feature 1..25: note = Description of sequence: Description: Name: CheckFP-inphage-R; ID: JG046
  source 1..25: mol_type = other DNA
agtgcttcttcttctggactaagtc

SEQ ID NO: 35 - DNA, 25 bp, synthetic construct
  misc_feature 1..25: note = Description of sequence: Description: Name: CheckFP-inphage-F; ID: JG047
  source 1..25: mol_type = other DNA
gtggtactatcagcctatccaaagg

SEQ ID NO: 36 - DNA, 49 bp, synthetic construct
  misc_feature 1..49: note = Description of sequence: Description: Name: AcrVIA1-XhoI-F; ID: JG065
  source 1..49: mol_type = other DNA
aggcccctcgagcctacgtaggaataaaaaatgatctactatataaaag

SEQ ID NO: 37 - DNA, 32 bp, synthetic construct
  misc_feature 1..32: note = Description of sequence: Description: Name: AcrVIA1-NdeI-R; ID: JG066
  source 1..32: mol_type = other DNA
cggccccatatgttaatttagctcctctttta

SEQ ID NO: 38 - DNA, 20 bp, synthetic construct
  misc_feature 1..20: note = Description of sequence: Description: Name: AcrVIA1-mid-F; ID: JG088
  source 1..20: mol_type = other DNA
tccgttcgctcccgatacga

SEQ ID NO: 39 - DNA, 73 bp, synthetic construct
  misc_feature 1..73: note = Description of sequence: Description: Name: pHERD-Op24NheI-F; ID: JG095
  source 1..73: mol_type = other DNA
gccttttgctggccttttgctgatcttaggccgtgagctggacataagttgaagacccgccactatatttagg

SEQ ID NO: 40 - DNA, 53 bp, synthetic construct
  misc_feature 1..53: note = Description of sequence: Description: Name: Op24H1-AcrVIA-R; ID: JG096
  source 1..53: mol_type = other DNA
cacttttaaatcttttatatagtagatcattttaaatacctttacgattatgg

SEQ ID NO: 41 - DNA, 50 bp, synthetic construct
  misc_feature 1..50: note = Description of sequence: Description: Name: Op24H1-AcrVIA-F; ID: JG097
  source 1..50: mol_type = other DNA
caagaaaccataatcgtaaaggtatttaaaatgatctactatataaaaga

SEQ ID NO: 42 - DNA, 50 bp, synthetic construct
  misc_feature 1..50: note = Description of sequence: Description: Name: AcrVIA-mCherry-R; ID: JG098
  source 1..50: mol_type = other DNA
caccattttttattcctacgtaggcctaggttaatttagctcctctttta

SEQ ID NO: 43 - DNA, 50 bp, synthetic construct
  misc_feature 1..50: note = Description of sequence: Description: Name: AcrVIA-mCherry-F; ID: JG099
  source 1..50: mol_type = other DNA
aattaacctaggcctacgtaggaataaaaaatggtgagcaagggcgagga

SEQ ID NO: 44 - DNA, 53 bp, synthetic construct
  misc_feature 1..53: note = Description of sequence: Description: Name: pHERD-gp54NheI-R; ID: JG100
  source 1..53: mol_type = other DNA
tgctctgcgaggctggccgataagctagatgtgggtcgcaatagagaacggag

SEQ ID NO: 45 - DNA, 50 bp, synthetic construct
  misc_feature 1..50: note = Description of sequence: Description: Name: pHERD-gp54end-F; ID: JG104
  source 1..50: mol_type = other DNA
gccttttgctggccttttgctcacataaggagcaagtcatcctagatgca

SEQ ID NO: 46 - DNA, 50 bp, synthetic construct
  misc_feature 1..50: note = Description of sequence: Description: Name: gp54end-gfp11-R; ID: JG105
  source 1..50: mol_type = other DNA
catgtggtcacgcgagccgcccgacccgccgtaccaggtacccggtgcat

SEQ ID NO: 47 - DNA, 25 bp, synthetic construct
  misc_feature 1..25: note = Description of sequence: Description: Name: KO39H2-F; ID: JG106
  source 1..25: mol_type = other DNA
attaacttatactggagcccttcgg

SEQ ID NO: 48 - DNA, 50 bp, synthetic construct
  misc_feature 1..50: note = Description of sequence: Description: Name: pHERD-KO39H2-R; ID: JG107
  source 1..50: mol_type = other DNA
tgctctgcgaggctggccgataagctagctttttagcagtgttctttgcc

SEQ ID NO: 49 - DNA, 25 bp, synthetic construct
  misc_feature 1..25: note = Description of sequence: Description: Name: gp54-mid-R; ID: JG050
  source 1..25: mol_type = other DNA
cagtggtcggagtccaatgtagatc

SEQ ID NO: 50 - DNA, 24 bp, synthetic construct
  misc_feature 1..24: note = Description of sequence: Description: Name: Check-recomb54-F; ID: JG108
  source 1..24: mol_type = other DNA
agtttgaggaccaagtgtaacacc

SEQ ID NO: 51 - DNA, 25 bp, synthetic construct
  misc_feature 1..25: note = Description of sequence: Description: Name: Check-KO54-R; ID: JG118
  source 1..25: mol_type = other DNA
gtgggttaattaggctacgtacgtg

SEQ ID NO: 52 - DNA, 50 bp, synthetic construct
  misc_feature 1..50: note = Description of sequence: Description: Name: pHERD-gp54NheI-F; ID: JG110
  source 1..50: mol_type = other DNA
gccttttgctggccttttgctcacataagctgcattttataaaaatactg

SEQ ID NO: 53 - DNA, 47 bp, synthetic construct
  misc_feature 1..47: note = Description of sequence: Description: Name: pHERD-gp54NheI-R; ID: JG111
  source 1..47: mol_type = other DNA
tgctctgcgaggctggccgataagctagttagtaccaggtacccggt

SEQ ID NO: 54 - DNA, 32 bp, synthetic construct
  misc_feature 1..32: note = Description of sequence: Description: Name: gp54-PstI-F; ID: JG127
  source 1..32: mol_type = other DNA
agcatcctgcagatggctgttaacgaaaacga

SEQ ID NO: 55 - DNA, 40 bp, synthetic construct
  misc_feature 1..40: note = Description of sequence: Description: Name: gp54-SacI-R; ID: JG128
  source 1..40: mol_type = other DNA
agcgtcgagctcttagtaccaggtacccggtgcattatag

SEQ ID NO: 56 - DNA, 50 bp, synthetic construct
  misc_feature 1..50: note = Description of sequence: Description: Name: AcrVIA-Op24H2-R; ID: JG112
  source 1..50: mol_type = other DNA
taacatccctatctaccgaggtatttcctattaatttagctcctctttta

SEQ ID NO: 57 - DNA, 55 bp, synthetic construct
  misc_feature 1..55: note = Description of sequence: Description: Name: AcrVIA-Op24H2-F; ID: JG113
  source 1..55: mol_type = other DNA
gcaaacaaattttaaaagaggagctaaattaataggaaatacctcggtagatagg

SEQ ID NO: 58 - DNA, 46 bp, synthetic construct
  misc_feature 1..46: note = Description of sequence: Description: Name: pHERD-Op24H2NheI-R; ID: JG114
  source 1..46: mol_type = other DNA
tgctctgcgaggctggccgataagctagctttctcatctgagttac

SEQ ID NO: 59 - DNA, 24 bp, synthetic construct
  misc_feature 1..24: note = Description of sequence: Description: Name: KO93H2-F; ID: JG119
  source 1..24: mol_type = other DNA
caaacataatgaggaacccttcgg

SEQ ID NO: 60 - DNA, 50 bp, synthetic construct
  misc_feature 1..50: note = Description of sequence: Description: Name: pHERD-KO93H2-R; ID: JG120
  source 1..50: mol_type = other DNA
tgctctgcgaggctggccgataagctagcataaaagcagctgattgttta

SEQ ID NO: 61 - DNA, 22 bp, synthetic construct
  misc_feature 1..22: note = Description of sequence: Description: Name: Check-KOgp93-R; ID: JG121
  source 1..22: mol_type = other DNA
agttagtacacgctgtgccgct

SEQ ID NO: 62 - DNA, 50 bp, synthetic construct
  misc_feature 1..50: note = Description of sequence: Description: Name: pHERD-gp39-NheI-F; ID: JG122
  source 1..50: mol_type = other DNA
gccttttgctggccttttgctcacataagattatattaatcacaatgagg

SEQ ID NO: 63 - DNA, 52 bp, synthetic construct
  misc_feature 1..52: note = Description of sequence: Description: Name: pHERD-gp39-NheI-R; ID: JG123
  source 1..52: mol_type = other DNA
tgctctgcgaggctggccgataagctagttaatcgaatacaagaccactact

SEQ ID NO: 64 - DNA, 52 bp, synthetic construct
  misc_feature 1..52: note = Description of sequence: Description: Name: pHERD-KO2412-H1Acr-F; ID: JG124
  source 1..52: mol_type = other DNA
gccttttgctggccttttgctcacataagttaaataaagataacttcttttc

SEQ ID NO: 65 - DNA, 40 bp, synthetic construct
  misc_feature 1..40: note = Description of sequence: Description: Name: KO2412-H1Acr-R; ID: JG125
  source 1..40: mol_type = other DNA
ccttttagttaatttaatttagctcctcttttaaaatttg

SEQ ID NO: 66 - DNA, 25 bp, synthetic construct
  misc_feature 1..25: note = Description of sequence: Description: Name: Check-KO2412-R; ID: JG126
  source 1..25: mol_type = other DNA
ggctaagtctttttctcgatactgg

SEQ ID NO: 67 - DNA, 53 bp, synthetic construct
  misc_feature 1..53: note = Description of sequence: Description: Name: pHERD-Op24NheI-F; ID: JG133
  source 1..53: mol_type = other DNA
gccttttgctggccttttgctcacataagtacgtgacggagcattcttaaata

SEQ ID NO: 68 - DNA, 50 bp, synthetic construct
  misc_feature 1..50: note = Description of sequence: Description: Name: pHERD-Op24NheI-R; ID: JG134
  source 1..50: mol_type = other DNA
tgctctgcgaggctggccgataagctagcccgaaggagccgctatatcag

SEQ ID NO: 69 - DNA, 51 bp, synthetic construct
  misc_feature 1..51: note = Description of sequence: Description: Name: Omk-capH1-GibsonF; ID: JG135
  source 1..51: mol_type = other DNA
gccttttgctggccttttgctcacataagatcgaaggcgcactatccgctg

SEQ ID NO: 70 - DNA, 57 bp, synthetic construct
  misc_feature 1..57: note = Description of sequence: Description: Name: Omk-capmuH1-GibsonR; ID: JG136
  source 1..57: mol_type = other DNA
gatcattttttattcctacgtagggctagcctaaggttttaagtcggcaagcttacc

SEQ ID NO: 71 - DNA, 50 bp, synthetic construct
  misc_feature 1..50: note = Description of sequence: Description: Name: pHERD-Op24InH1-F; ID: JG137
  source 1..50: mol_type = other DNA
gccttttgctggccttttgctcacataagggagcattcttaaataaatta

SEQ ID NO: 72 - DNA, 50 bp, synthetic construct
  misc_feature 1..50: note = Description of sequence: Description: Name: Op24InH1-RBS-R; ID: JG138
  source 1..50: mol_type = other DNA
gatcattgtatattcctatttgtaatctcgagcgtaacgaacactatgtt

SEQ ID NO: 73 - DNA, 46 bp, synthetic construct
  misc_feature 1..46: note = Description of sequence: Description: Name: gp53RBS-AcrVIA1-F; ID: JG139
  source 1..46: mol_type = other DNA
ctcgagattacaaataggaatatacaatgatctactatataaaaga

SEQ ID NO: 74 - DNA, 50 bp, synthetic construct
  misc_feature 1..50: note = Description of sequence: Description: Name: AcrVIA1-Op24InH2-R; ID: JG140
  source 1..50: mol_type = other DNA
cgctataatctgctatcagcagatcctaggttaatttagctcctctttta

SEQ ID NO: 75 - DNA, 44 bp, synthetic construct
  misc_feature 1..44: note = Description of sequence: Description: Name: AcrVIA1-Op24InH2-F; ID: JG141
  source 1..44: mol_type = other DNA
aggagctaaattaacctaggatctgctgatagcagattatagcg

SEQ ID NO: 76 - DNA, 46 bp, synthetic construct
  misc_feature 1..46: note = Description of sequence: Description: Name: pHERD-Op24InH2-R; ID: JG142
  source 1..46: mol_type = other DNA
tgctctgcgaggctggccgataagctaggttgtaacggttccgtag

SEQ ID NO: 77 - DNA, 50 bp, synthetic construct
  misc_feature 1..50: note = Description of sequence: Description: Name: pHERD-KOgp146H1-F; ID: JG146
  source 1..50: mol_type = other DNA
gccttttgctggccttttgctcacataagagctattgcaaatgcggtacc

SEQ ID NO: 78 - DNA, 56 bp, synthetic construct
  misc_feature 1..56: note = Description of sequence: Description: Name: KOgp146H1-AcrR; ID: JG147
  source 1..56: mol_type = other DNA
tagatcatttttatttccttgctgtataagaacattatactacttcctcaagttcg

SEQ ID NO: 79 - DNA, 25 bp, synthetic construct
  misc_feature 1..25: note = Description of sequence: Description: Name: Check-gp146edit-R; ID: JG148
  source 1..25: mol_type = other DNA
aatctatcccagttcaagctaagcc

SEQ ID NO: 80 - DNA, 45 bp, synthetic construct
  misc_feature 1..45: note = Description of sequence: Description: Name: gp145RBS-AcrVIA1-F; ID: JG152
  source 1..45: mol_type = other DNA
ttcttatacagcaaggaaataaaaatgatctactatataaaagat

SEQ ID NO: 81 - DNA, 42 bp, synthetic construct
  misc_feature 1..42: note = Description of sequence: Description: Name: AcrVIA1-Op63InH2-R; ID: JG153
  source 1..42: mol_type = other DNA
ggagggcaatatgtcgttttaatttagctcctcttttaaaat

SEQ ID NO: 82 - DNA, 42 bp, synthetic construct
  misc_feature 1..42: note = Description of sequence: Description: Name: AcrVIA1-Op63InH2-F; ID: JG154
  source 1..42: mol_type = other DNA
aaagaggagctaaattaaaacgacatattgccctcccttcgg

SEQ ID NO: 83 - DNA, 55 bp, synthetic construct
  misc_feature 1..55: note = Description of sequence: Description: Name: pHERD-Op63InH2-R; ID: JG155
  source 1..55: mol_type = other DNA
tgctctgcgaggctggccgataagctagattccatttattattcaattaatagct

SEQ ID NO: 84 - DNA, 54 bp, synthetic construct
  misc_feature 1..54: note = Description of sequence: Description: Name: pMMB-gp146-GbF; ID: JG156
  source 1..54: mol_type = other DNA
ggaaacagaattaattaagcttgcatgcctgcagatggcttctgcatttgaaga

SEQ ID NO: 85 - DNA, 51 bp, synthetic construct
  misc_feature 1..51: note = Description of sequence: Description: Name: pMMB-gp146-GbR; ID: JG157
  source 1..51: mol_type = other DNA
ccgccaaaacagccaagctgaattcgagctcttaaataacagcacctttgg

SEQ ID NO: 86 - DNA, 56 bp, synthetic construct
  misc_feature 1..56: note = Description of sequence: Description: Name: AcrVIA1-KO241H2-F; ID: JG160
  source 1..56: mol_type = other DNA
caaacaaattttaaaagaggagctaaattaataagagcatatggccctcccttcgg

SEQ ID NO: 87 - DNA, 50 bp, synthetic construct
  misc_feature 1..50: note = Description of sequence: Description: Name: pHERD-KO241H2-R; ID: JG161
  source 1..50: mol_type = other DNA
tgctctgcgaggctggccgataagctagtaatagtggtattaacttgtag

SEQ ID NO: 88 - DNA, 28 bp, synthetic construct
  misc_feature 1..28: note = Description of sequence: Description: Name: Check-KOgp241-R; ID: JG162
  source 1..28: mol_type = other DNA
cgggtgtaatcgtacgtagatcaatcgc

SEQ ID NO: 89 - DNA, 45 bp, synthetic construct
  misc_feature 1..45: note = Description of sequence: Description: Name: KO2412-H2-F; ID: JG166
  source 1..45: mol_type = other DNA
gaggagctaaattaaattaactaaaaggtaaaaaaatgaataatc

SEQ ID NO: 90 - DNA, 50 bp, synthetic construct
  misc_feature 1..50: note = Description of sequence: Description: Name: pHERD-KO2412-H2-R; ID: JG167
  source 1..50: mol_type = other DNA
tgctctgcgaggctggccgataagctagcatccttattatggaagctata

SEQ ID NO: 91 - DNA, 24 bp, synthetic construct
  misc_feature 1..24: note = Description of sequence: Description: Name: Check-PaM24-R; ID: JG168
  source 1..24: mol_type = other DNA
ctcgtggtgatgttaaccctaggg

SEQ ID NO: 92 - DNA, 25 bp, synthetic construct
  misc_feature 1..25: note = Description of sequence: Description: Name: Check-PaM24-F; ID: JG169
  source 1..25: mol_type = other DNA
caactctgaattccgaactgaggcc

SEQ ID NO: 93 - DNA, 26 bp, synthetic construct
  misc_feature 1..26: note = Description of sequence: Description: Name: Check-KOgp93-F; ID: JG170
  source 1..26: mol_type = other DNA
actcttgtatatccaatctgtagcgg

SEQ ID NO: 94 - DNA, 53 bp, synthetic construct
  misc_feature 1..53: note = Description of sequence: Description: Name: gp39-SacI-F; ID: JG173
  source 1..53: mol_type = other DNA
acccatgggatctgataagaattcgagctcatgatgtctaaagtaaaaactcg

SEQ ID NO: 95 - DNA, 54 bp, synthetic construct
  misc_feature 1..54: note = Description of sequence: Description: Name: gp39-KpnI-R; ID: JG174
  source 1..54: mol_type = other DNA
caggtcgactctagaggatccccgggtaccttaatcgaatacaagaccactact

SEQ ID NO: 96 - DNA, 60 bp, synthetic construct
  misc_feature 1..60: note = Description of sequence: Description: Name: pHERD-KOop42H1-F; ID: JG175
  source 1..60: mol_type = other DNA
gccttttgctggccttttgctcacataagtaacctaccttcttttactaatgcagaatag

SEQ ID NO: 97 - DNA, 56 bp, synthetic construct
  misc_feature 1..56: note = Description of sequence: Description: Name: KOop42H1-R; ID: JG176
  source 1..56: mol_type = other DNA
cttttaaatcttttatatagtagatcatttttctcttcctatatgggtttgcagag

SEQ ID NO: 98 - DNA, 50 bp, synthetic construct
  misc_feature 1..50: note = Description of sequence: Description: Name: pHERD-gfp10-GbF; ID: JG178
  source 1..50: mol_type = other DNA
acccatgggatctgataagaattcgagctcatggtgagcaagggcgagga

SEQ ID NO: 99 - DNA, 50 bp, synthetic construct
  misc_feature 1..50: note = Description of sequence: Description: Name: pHERD-gfp10-GbR; ID: JG179
  source 1..50: mol_type = other DNA
ggccagtgccaagcttgcatgcctgcagcttctcgttggggtctttgctc

SEQ ID NO: 100 - DNA, 25 bp, synthetic construct
  misc_feature 1..25: note = Description of sequence: Description: Name: gfp11-mid-F; ID: JG180
  source 1..25: mol_type = other DNA
tggtccttcatgagtatgtaaatgc

SEQ ID NO: 101 - DNA, 22 bp, synthetic construct
  misc_feature 1..22: note = Description of sequence: Description: Name: Check-KOgp39-R; ID: JG181
  source 1..22: mol_type = other DNA
cttcagcccatttgtctacccg

SEQ ID NO: 102 - DNA, 54 bp, synthetic construct
  misc_feature 1..54: note = Description of sequence: Description: Name: gp93-mNGH1-Gb-F; ID: JG182
  source 1..54: mol_type = other DNA
gccttttgctggccttttgctcacataagaatccgaagactattgcagacttca

SEQ ID NO: 103 - DNA, 56 bp, synthetic construct
  misc_feature 1..56: note = Description of sequence: Description: Name: gp93-mNGH1-Gb-R; ID: JG183
  source 1..56: mol_type = other DNA
ctatatgggtttgcggttcctcattatgtttgttattacttgtacagctcgtccat

SEQ ID NO: 104 - DNA, 25 bp, synthetic construct
  misc_feature 1..25: note = Description of sequence: Description: Name: Check-KOop42-F; ID: JG186
  source 1..25: mol_type = other DNA
caccaataaagtcaccagtacgtcc

SEQ ID NO: 105 - DNA, 25 bp, synthetic construct
  misc_feature 1..25: note = Description of sequence: Description: Name: gp93-mid-F; ID: JG187
  source 1..25: mol_type = other DNA
gccctagctgattggaatcctgacg

SEQ ID NO: 106 - DNA, 60 bp, synthetic construct
  misc_feature 1..60: note = Description of sequence: Description: Name: Lse_crRNA_V3_Top; ID: oSDM455
  source 1..60: mol_type = other DNA
actctctactgtttctccatggtaagagactacctctatatgaaagaggactaaaaccga

SEQ ID NO: 107 - DNA, 60 bp, synthetic construct
  misc_feature 1..60: note = Description of sequence: Description: Name: Lse_crRNA_V3_Bot; ID: oSDM456
  source 1..60: mol_type = other DNA
cgggtaccgagctcgaattcgagaccgtacgtacgtggtctcggttttagtcctctttca

SEQ ID NO: 108 - DNA, 22 bp, synthetic construct
  misc_feature 1..22: note = Description of sequence: Description: Name: p30T_TSS-R; ID: oSDM457
  source 1..22: mol_type = other DNA
atggagaaacagtagagagttg

SEQ ID NO: 109 - DNA, 18 bp, synthetic construct
  misc_feature 1..18: note = Description of sequence: Description: Name: p30T_TSS-F; ID: oSDM458
  source 1..18: mol_type = other DNA
gaattcgagctcggtacc

SEQ ID NO: 110 - DNA, 78 bp, synthetic construct
  misc_feature 1..78: note = Description of sequence: Description: Name: LseCas13 crRNA V2 F; ID: oSDM465
  source 1..78: mol_type = other DNA
actctctactgtttctccatggtaagagactacctctatatgaaagaggactaaaaccgagaccacgtacgtacggtc

SEQ ID NO: 111 - DNA, 78 bp, synthetic construct
  misc_feature 1..78: note = Description of sequence: Description: Name: LseCas13 crRNA V2 R; ID: oSDM466
  source 1..78: mol_type = other DNA
cgggtaccgagctcgaattcgtttttgtggagattcatattctccaagtctcttaccggagaccgtacgtacgtggtc

SEQ ID NO: 112 - DNA, 106 bp, synthetic construct
  misc_feature 1..106: note = Description of sequence: Description: WT Phi Kappa Zeta
  source 1..106: mol_type = other DNA
gctgatcttaagccgtgagctggaaagggttaccgttatctagaaaatgggactatccgttatttggaagacggtaatgcccggaagttagaagagtaacagacat

SEQ ID NO: 113 - DNA, 79 bp, Listeriophage Phi-LS46
  misc_feature 1..79: note = Description of sequence: Description: Escaper 1
  source 1..79: mol_type = genomic DNA
gctgatcttaagccgtgaaatgggactatccgttatttggaagacggtaatgcccggaagttagaagagtaacagacat

SEQ ID NO: 114 - DNA, 76 bp, synthetic construct
  misc_feature 1..76: note = Description of sequence: Description: Escaper 2
  source 1..76: mol_type = other DNA
gctgatcttaagccgtgagggactatccgttatttggaagacggtaatgcccggaagttagaagagtaacagacat

SEQ ID NO: 115 - DNA, 37 bp, synthetic construct
  misc_feature 1..37: note = Description of sequence: Description: Escaper 3
  source 1..37: mol_type = other DNA
gctgatcttaagccgtagttagaagagtaacagacat